(19) United States
(12) Patent Application Publication
(10) Pub. No.: US 2024/0293600 A1
(43) Pub. Date: Sep. 5, 2024

(54) COMPOSITIONS, METHODS, AND DEVICES FOR SUSTAINED RELEASE OF AN AGENT

(71) Applicant: Board of Supervisors of Louisiana State University and Agricultural and Mechanical College, Baton Rouge, LA (US)

(72) Inventors: Tammy R. Dugas, Baton Rouge, LA (US); Cristina Sabliov, Baton Rouge, LA (US); Carlos Astete, Baton Rouge, LA (US)

(21) Appl. No.: 18/571,757

(22) PCT Filed: Jun. 21, 2022
(86) PCT No.: PCT/US2022/073054

§ 371 Date: Dec. 19, 2023

Related U.S. Application Data
(60) Provisional application No. 63/202,673, filed on Jun. 21, 2021.

(51) Int. Cl. A61L 27/54; A61L 27/18; A61L 29/06; A61L 29/16; A61L 31/06; A61L 31/16

(52) U.S. Cl. CPC A61L 27/54; A61L 27/18; A61L 29/06; A61L 29/16; A61L 31/06; A61L 31/16; A61L 2300/416; A61L 2300/624; A61L 2400/12

(57) ABSTRACT

The present disclosure provides for compositions, methods of making the composition, and devices (e.g., vascular grafts, self-expandable stents, balloon-expandable stents, and stent-grafts) having the composition disposed thereof. The composition can include a polymer nanoparticle (e.g., charged or uncharged) made of polymers that have one or more types of agents covalently bonded to the polymer. In this regard, the one or more types of agents are not physically encapsulated by the polymer, rather are covalently bonded to the polymer that makes up the polymeric nanoparticle. In another aspect, an agent can also be encapsulated in the polymer of the polymeric nanoparticle. In another embodiment, the composition includes two types of polymeric nanoparticles, where a first polymeric nanoparticle has at least one agent covalently bonded to the polymer of the first polymeric nanoparticle and where the composition can also include a second polymeric nanoparticle encapsulating an agent.

### CLAIM OF PRIORITY TO RELATED APPLICATION

This application claims priority to co-pending U.S. provisional application entitled “COMPOSITIONS, METHODS AND DEVICES FOR SUSTAINED RELEASE OF AN AGENT” having Ser. No. 63/202,673, filed on Jun. 21, 2021, which is entirely incorporated herein by reference.

### FEDERAL FUNDING

This invention was made with government support under R41 HL142403 awarded by the National Institutes of Health. The government has certain rights in the invention.

### BACKGROUND

Peripheral artery disease (PAD) is a systemic vascular disease of the legs that results in a blockage of blood flow from the heart to the lower extremities. Now one of the most common causes of mortality in the U.S., the first line of therapy for PAD is to mechanically open the blockages using balloon angioplasty. Coating the balloons with antiproliferative agents can potentially reduce vessel re-narrowing, or restenosis after surgical intervention, but current drug-coated balloons releasing chemotherapy agents like paclitaxel have in some cases shown increased mortality long-term. Thus, there is a need in the art to address these and/or other related issues or problems.

### SUMMARY

The present disclosure provides for compositions, methods of making the composition, and devices (e.g., vascular grafts, self-expandable stents, balloon-expandable stents, and stent-grafts) having the composition disposed thereof. The composition, as well as therapeutic compositions, can include a polymer nanoparticle (e.g., charged or uncharged) made of polymers that have one or more types of agents covalently bonded to the polymer. In this regard, one or more types of agents are not physically encapsulated by the polymer, rather are covalently bonded to the polymer that makes up the polymeric nanoparticle. In another aspect, an agent can also be encapsulated in the polymer of the polymeric nanoparticle. In another embodiment, the composition includes two types of polymeric nanoparticles, where a first polymeric nanoparticle has at least one agent covalently bonded to the polymer of the first polymeric nanoparticle and where the composition can also include a second polymeric nanoparticle encapsulating an agent.

The present disclosure provides for compositions, as well as therapeutic compositions, comprising a polymeric nanoparticle made of a polymer having an agent is covalently bonded to the polymer. The polymer can be selected from the group consisting of poly(lactide), poly(glycolide), poly(lactide-co-glycolide), poly(caprolactone), poly(lactide-co-caprolactone), poly(glycolide-co-caprolactone), and poly(D,L-lactide-co-glycolide-co-ε-caprolactone). The agent can be selected from the group consisting of resveratrol, pharmaceutically acceptable salts, and pharmaceutically acceptable derivatives thereof, or quercetin, pharmaceutically acceptable salts, and pharmaceutically acceptable derivatives thereof, rhamnazin, pharmaceutically acceptable salts, and pharmaceutically acceptable derivatives thereof, or a combination of any of these.

The present disclosure provides for compositions, as well as therapeutic compositions, comprising a first polymeric nanoparticle made of a first polymer having a first agent covalently bonded to the polymer and a second polymeric nanoparticle made of a second polymer, where the second polymer encapsulates a second agent. The first polymer and the second polymer can independently be selected from the group consisting of poly(lactide), poly(glycolide), poly(lactide-co-glycolide), poly(caprolactone), poly(lactide-co-caprolactone), poly(glycolide-co-caprolactone), and poly(D,L-lactide-co-glycolide-co-ε-caprolactone). The first agent and the second agent can independently be selected from the group consisting of resveratrol, pharmaceutically acceptable salts, and pharmaceutically acceptable derivatives thereof, or quercetin, pharmaceutically acceptable salts, and pharmaceutically acceptable derivatives thereof, rhamnazin, pharmaceutically acceptable salts, and pharmaceutically acceptable derivatives thereof, or a combination of any of these.

The present disclosure provides for devices, comprising: a drug coated device having an outer surface; and a nanoparticle coating the outer surface of device comprising a composition as described above and herein. The device can be a balloon catheter or a vascular graft, for example.

The present disclosure provides for methods for treating a vascular disease comprising treating a subject with the device as provided above and herein.

The present disclosure provides for kits comprising a device as provided above and herein and instructions for using the device to treat a vascular disease.

The present disclosure provides for kits comprising a composition as provided above and herein, a device, instructions for coating the on a device.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of this disclosure. Moreover, in the drawings, like reference numerals designate corresponding parts throughout the several views.

FIGS. 1A and 1B illustrate representative balloon coating images illustrating methods used for assessing uniformity. Gray circles indicate points of reference for recording the location of segments examined (yellow boxes). Each picture has a width of 1973 μm and a height of 1457 μm, and the illustration highlights two distinctive areas (left-right) for histogram-based fluorescence analysis. In FIG. 1A (top row), balloon 1 coated with 5 layers (5LYR1), the circle is 5941 μm from proximal end, and in FIG. 1B (bottom row), balloon 2 coated with 15 layers (15LYR2), the circle is 5504 μm from the proximal end.

FIG. 2 illustrates examples of stitched images used to reconstruct the balloons for image analysis. Shown is balloon 2 coated with 5 (A), 10 (B), 15 (C), and 20 layers (D) of pNp coating, visible+fluorescent (light gray) green overlay (at 75% opacity).

FIG. 3A-3D illustrate: TEM images of (FIG. 3A) empty pNP (pNP(E)); (FIG. 3B) pNP with entrapped rhamnazin (3′,7-dimethylquercetin, DMQ) (pNP(eDMQ)) at magnification of 50,000×; (FIG. 3C) pNP with entrapped quercetin (pNP(eQ)); and (FIG. 3D) pNP with covalently attached quercetin (pNP(cQ)) at magnification of 80,000×.

FIG. 4 illustrates a plot that measures of percent drug release from pNP containing entrapped quercetin (pNP(eQ)), covalently attached quercetin (pNP(cQ)) and entrapped rhamnazin (3′,7-dimethylquercetin, DMQ) (pNP(cDMQ)). Protracted release was observed mainly for pNPs(cQ). Data are means±SD for n=3.

FIG. 5 illustrates a graph that shows rat aortic smooth muscle cells loaded for 2 hours with empty pNPs (pNPs(E)), entrapped quercetin (pNPs(eQ)), covalent quercetin (pNPs(cQ)) and entrapped rhamnazin (3′,7-dimethylquercetin, DMQ) (pNPs(eDMQ)) exhibit reduced rates of cell proliferation at 24, 48 and 72 hours after washing. DNA synthesis was assessed by determining the incorporation of BrDU compared to control cells treated with no pNPs. Data are means±SD for n=8. Two-way ANOVA revealed a significant effect of treatment. *Indicates significance compared to controls for the same time point, revealed using Dunnett's post-hoc test. Dotted line represents the response for control cells treated with no pNPs, denoted as 100%.

FIG. 6 illustrates a graph that shows pNPs(cQ) exhibit a reduced ability to bind to rat aortic smooth muscle cells but their binding is resistant to washing and calcification. pNP suspensions at 2 mg/mL were allowed to bind to cells for 2 hours before washing with buffer. Some sets of cells were subjected to a calcification treatment prior to pNP exposure. Gray (green) fluorescence determined before and after washing was normalized to protein in the well. Data are means±SD for n=9. Three-way ANOVA revealed a significant effect of pNP treatment, calcification and washing. *Indicates significance compared to empty nanoparticles (pNPs(E)) for the same cell treatment. #Represents significance compared to unwashed wells for the same pNP treatment.

FIG. 7 illustrates representative images of green fluorescence within rat aortic smooth muscle cells exposed to pNPs containing quercetin. pNP suspensions at 2 mg/mL were allowed to bind to cells for two hours before washing with buffer. Some sets of cells were subjected to a calcification treatment prior to pNP exposure (right panel). Gray (green) fluorescence was imaged after washing. Yellow bar=100 μm.

FIGS. 8A and 8B illustrate images of pNPs containing covalently attached quercetin exhibit binding to clusters (indicated by arrows) within calcified smooth muscle cell cultures that were visible in both brightfield (FIG. 8A) and fluorescence (FIG. 8B) images.

FIGS. 9A and 9B illustrate fluorescence imaging revealed that ultrasonic coating with pNPs entrapping polyphenols yields uniform coatings. FIG. 9A shows the overall mean fluorescence and corresponding standard deviations for n=8 samples (balloons ultrasonically coated). Data illustrated in the graph represent mean fluorescence ±SD (in unit of brightness per μm²). Maximum brightness for a 16-bit image is 65535, corresponding to 12117.42 per μm². FIG. 9B illustrates the percent of balloon area that has pixels with fluorescence intensity within a ±1-SD of the mean fluorescence. Data represent means±SD for n=8 samples. Higher value indicates better uniformity.

FIGS. 10A and 10B illustrate the amount of quercetin nanoparticles (pNPs) and quercetin (Q) in balloon coating. FIG. 8A illustrate that pNPs were eluted from the balloons using organic solvent and pNP load was determined gravimetrically. Total loading in μg was normalized to balloon areas. Data represent means±SD for 5 measures per balloon. FIG. 10B illustrates that pNPs were eluted from the balloons using organic solvent and the Q content was determined using HPLC. Data represent means±SD for n=3-4 replicate measures/balloon.

FIG. 11 is a graph that illustrates the efficacy of a resveratrol:quercetin-eluting stent (RQ-DES) compared to a bare metal stent (BMS). Mini-stents were coated with 50:25 R:Q (RQ1) and 100:50 μg/cm² R:Q (RQ2) and were implanted into male and female rats. At 28 d, stented arteries were analyzed for intimal areas. n=6//gp. *p<0.05 compared to unstented arteries. #p<0.05 compared to BMS.

FIGS. 12A-12C illustrate that RQ-DES accelerates re-endothelialization. FIG. 12A illustrates photomicrographs of whole-mounted carotid arteries 10 d after stenting with either BMS or RQ2-coated stent. Injured endothelium was identified using Evans blue dye. FIG. 12B illustrates the arterial dye content was quantified by extracting and measuring its absorbance. Data were normalized to tissue weight. FIG. 12C illustrates the re-endothelialized area normalized to stented area. Data are means±SD. *p<0.05.

### DETAILED DESCRIPTION

The present disclosure provides for a composition, as well as therapeutic compositions, that can be applied to a device (e.g., balloon catheter or other medical device), where an agent can have a sustained release profile. In an aspect, one or more types of agents, such as a polyphenol (e.g., resveratrol, quercetin, methoxylated quercetin and rhamnazin (3′,7-dimethylquercetin, DMQ)) can be covalently bonded to a polymer in a nanoparticle (e.g., PLGA nanoparticle), which has the characteristic of sustained release over the course of days. Optionally, another type of agent (e.g., a polyphenol) can be encapsulated by the polymer of the nanoparticle in addition to the agent covalently bonded to the polymer. In this regard, one type of agent can be covalently bonded to the polymer while another type of agent can be encapsulated by the polymer. In another aspect, the composition can include two types of polymeric nanoparticles. For example, the composition, as well as therapeutic compositions, can include a first polymeric nanoparticle that has at least one agent covalently bonded to the polymer of the first polymeric nanoparticle and the composition can also include a second polymeric nanoparticle encapsulating an agent. In each instance, the polymeric nanoparticle can be neutral or charged. Each of these compositions, as well as therapeutic compositions, can be applied to a device such as balloon catheters, vascular grafts, self-expandable stents, balloon-expandable stents, and stent-grafts to deliver one or more agents and can do so to provide a sustained release profile over a number of days for the agent that is covalently bonded to the polymer.

In an aspect, this can be compared to standard formulations where all of the agents are physically entrapped in the polymeric matrix as opposed to having at least one type of agent covalently bonded to the polymer of the polymeric nanoparticle.

The present disclosure also provides for the synthesis of polymer nanoparticles covalently bonded to an agent, (e.g., PLGA-polyphenol), where the agent is covalently bonded to the polymer in a two-step acylation reaction. Then the polymer including the agent is formed into a polymeric nanoparticle.

The present disclosure can be understood more readily by reference to the following detailed description of the disclosure and the Examples included therein, in which some, but not all possible embodiments are shown. Indeed, disclosures may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed compositions and methods belong. In this specification and in the claims which follow, reference will be made to a number of terms which shall be defined herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Prior to describing the various embodiments, the following definitions are provided and should be used unless otherwise indicated.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

As used herein, nomenclature for compounds, including organic compounds, can be given using common names, IUPAC, IUBMB, or CAS recommendations for nomenclature.

When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, “comprising” is to be interpreted as specifying the presence of the stated features, integers, steps, or components as referred to, but does not preclude the presence or addition of one or more features, integers, steps, or components, or groups thereof. Additionally, the term “comprising” is intended to include examples encompassed by the terms “consisting essentially of” and “consisting of.” Similarly, the term “consisting essentially of” is intended to include examples encompassed by the term “consisting of.” As used herein, the terms “by”, “comprising,” “comprises”, “comprised of,” “including,” “includes,” “included,” “involving,” “involves,” “involved,” and “such as” are used in their open, non-limiting sense.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Reference to “a” chemical compound refers one or more molecules of the chemical compound, rather than being limited to a single molecule of the chemical compound. Furthermore, the one or more molecules may or may not be identical, so long as they fall under the category of the chemical compound. Thus, for example, “a” PLGA is interpreted to include one or more polymer molecules of the PLGA, where the polymer molecules may or may not be identical (e.g., different molecular weights and/or isomers).

It should be noted that ratios, concentrations, amounts, and other numerical data can be expressed herein in a range format. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.4%, 3.2%, and 4.4%) within the indicated range.

As used herein, the terms “about,” “approximate,” and “at or about” mean that the amount or value in question can be the exact value designated or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

References in the specification and concluding claims to parts by weight of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein the terms “weight percent,” “wt %,” and “wt. %,” which can be used interchangeably, indicate the percent by weight of a given component based on the total weight of the composition, unless otherwise specified. That is, unless otherwise specified, all wt % values are based on the total weight of the composition. It should be understood that the sum of wt % values for all components in a disclosed composition or formulation are equal to 100.

Compounds are described using standard nomenclature. For example, any position not substituted by any indicated group is understood to have its valence filled by a bond as indicated, or a hydrogen atom. A dash (“-”) that is not between two letters or symbols is used to indicate a point of attachment for a substituent. For example, —CHO is attached through carbon of the carbonyl group. Unless defined otherwise, technical and scientific terms used herein have the same meaning as is commonly understood by one of skill in the art to which this invention belongs.

The term “alkyl group” as used herein is a branched or unbranched saturated hydrocarbon group of 1 to 24 carbon atoms, such as methyl, ethyl, n propyl, isopropyl, n butyl, isobutyl, t butyl, pentyl, hexyl, heptyl, octyl, decyl, tetradecyl, hexadecyl, eicosyl, tetracosyl and the like. A “lower alkyl” group is an alkyl group containing from one to six carbon atoms.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As used herein, the terms “number average molecular weight” or “Mₙ” can be used interchangeably, and refer to the statistical average molecular weight of all the polymer chains in the sample and is defined by the formula:

$${M}_{n} = \frac{\sum {N}_{i} {M}_{i}}{\sum {N}_{i}} ,$$

where Mᵢ is the molecular weight of a chain and Nᵢ is the number of chains of that molecular weight. Mₙ can be determined for polymers, e.g., polycarbonate polymers, by methods well known to a person having ordinary skill in the art using molecular weight standards, e.g. polycarbonate standards or polystyrene standards, preferably certified or traceable molecular weight standards.

As used herein, a “polymer” refers to a molecule comprised of repeating “constitutional units.” The constitutional units derive from the reaction of monomers. The constitutional units themselves can be the product of the reactions of other compounds. A polymer may be derived from the polymerization of two or more different monomers and therefore may comprise two or more different constitutional units. Such polymers are referred to as “copolymers.” “Terpolymers” are a subset of “copolymers” in which there are three different constitutional units. Those skilled in the art, given a particular polymer, will readily recognize the constitutional units of that polymer and will readily recognize the structure of the monomer from which the constitutional units derive. Polymers may be straight chain, branched chain, star-like or dendritic. One polymer may be attached (grafted) onto another polymer. The constitutional units of polymers may be randomly disposed along the polymer chain, may be present as discrete blocks, may be so disposed as to form gradients of concentration along the polymer chain, or a combination thereof. Polymers may be cross-linked to form a network.

As used herein, the term “units” can be used to refer to individual (co)monomer units such that, for example, glycolide repeat units refers to individual styrene (co)monomer units in the polymer. In addition, the term “units” can be used to refer to polymeric block units such that, for example, “glycolide repeating units” can also refer to glycolide blocks; “units of polylactide” refers to block units of polylactide; “units of polyglycolide” refers to block units of polyglycolide; and so on. Such use will be clear from the context.

The term “copolymer” refers to a polymer having two or more monomer species, and includes terpolymers (i.e., copolymers having three monomer species).

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

The term “contacting” as used herein refers to bringing a disclosed compound and a cell, a target protein, or other biological entity together in such a manner that the compound can affect the activity of the target, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “therapeutic agent” or “therapeutic composition” also refers to pharmaceutically acceptable, pharmacologically active derivatives of those therapeutic agent disclosed herein, such as resveratrol and quercetin, including, but not limited to, salts, esters, amides, hydrates, solvates, and the like.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” or “therapeutic composition” include any synthetic or naturally occurring biologically active compound or composition of matter which, when administered to an organism (human or nonhuman animal), induces a desired pharmacologic, immunogenic, and/or physiologic effect by local and/or systemic action.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, a “cardiovascular disease” is a disease, condition, or disorder that impacts the heart, circulatory system, or both the heart and the circulatory system. The circulatory system includes the cardiovascular system, and the lymphatic system. The lymphatic system distributes lymph. The cardiovascular system is a system of blood vessels, primarily arteries and veins, which transport blood to and from the heart, brain and peripheral organs such as, without limitation, the arms, legs, kidneys and liver. The coronary artery system supplies blood to the heart. The carotid artery system supplies blood to the brain. The peripheral vascular system carries blood to (via arteries) and from (via veins) the peripheral organs such as, without limitation, the hands, legs, kidneys and liver. The coronary artery system, carotid artery system, and the peripheral vascular system which includes the peripheral artery system are sub-systems of the cardiovascular system.

As used herein, a “vascular disease” generally refers to a disease, condition, or disorder that impacts the circulatory system. In particular “vascular disease” includes a disease, disorder, or condition of the coronary system, the carotid system and the peripheral vascular system. Vascular disease can include vascular calcification.

“Vascular diseases” are a subset of “cardiovascular diseases.”

Examples of cardiovascular diseases include diseases of the heart which include, but are not limited to, heart valve disease, arrhythmia, heart failure, and congenital heart disease, and vascular diseases which include, but are not limited to atherosclerosis, thrombosis, restenosis, hemorrhage, vascular dissection or perforation, vulnerable plaque, chronic total occlusion, claudication, anastomotic proliferation for vein and artificial grafts, peripheral artery disease, carotid artery disease, coronary artery disease, aneurysm, renal (kidney) artery disease, raynaud's syndrome, buerger's disease, peripheral venous disease, varicose veins, blood clots in the veins, blood clotting disorders, and lymphedema.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, vascular grafts, self-expandable stents, balloon-expandable stents, and stent-grafts.

One type of implantable medical device is a stent. Stents are implantable medical devices that are generally cylindrically shaped, and function to hold open, and sometimes expand, a segment of a blood vessel or other lumen or vessel in a patient's body when the vessel is narrowed or closed due to diseases or disorders including, without limitation, coronary artery disease, carotid artery disease and peripheral arterial disease. A stent can be used in, without limitation, neuro, carotid, coronary, pulmonary, renal, biliary, iliac, femoral and popliteal, as well as other peripheral vasculatures, as well as other bodily lumens. A stent can be used in the treatment or prevention of vascular disorders, as well as other disorders. For a stent, the “outer surface” includes the luminal surface which faces the lumen interior, the abluminal surface which faces the lumen wall, and sidewall surfaces, if present, which connect the abluminal and luminal surfaces.

A “catheter” is a thin, flexible tube for insertion into a natural body cavity, duct, or vessel, and may be used to introduce or remove fluid, to distend the vessel, or to hold open the vessel or cavity.

A “vascular catheter” is an example of an insertable medical device. A vascular catheter is a thin, flexible tube with a manipulating means at one end, which remains outside the patient's body, and an operative device at or near the other end, which is inserted into the patient's artery or vein. The catheter may be used for the introduction of fluids, often containing drugs, to the target site. The catheter may be used to deliver a stent to the target site, or may be used to deliver a balloon used in angioplasty. The catheter may perform multiple functions.

As used herein, a “balloon” comprises a relatively thin, flexible material, forming a tubular membrane, and is usually associated with a vascular catheter. When positioned at a particular location in a patient's vessel can be expanded or inflated to an outside diameter that is essentially the same as the inside or luminal diameter of the vessel in which it is placed. Balloons may be inflated, without limitation, using a liquid medium such as water or normal saline solution (where saline means including salt, typically sodium chloride), that is, saline that is essentially isotonic with blood.

A “balloon catheter” refers to a medical device which is a system of a catheter with a balloon at the end of the catheter.

With respect to a DCB catheter balloon, the “outer surface” is meant any surface however spatially oriented that is in contact with a bodily tissue, such as a vessel wall, or fluid.

### Discussion

The present disclosure provides for compositions, methods of making the composition, and devices (e.g., vascular grafts, self-expandable stents, balloon-expandable stents, and stent-grafts) having the composition disposed thereof. The composition (as well as therapeutic compositions including the composition) can include a polymer nanoparticle (e.g., charged or uncharged) made of polymers that have one or more types of agents covalently bonded to the polymer. In this regard, the one or more types of agents are not physically encapsulated by the polymer, rather are covalently bonded to the polymer that makes up the polymeric nanoparticle. In another aspect, an agent can also be encapsulated in the polymer of the polymeric nanoparticle. In another embodiment, the composition, as well as therapeutic compositions, includes two types of polymeric nanoparticles, where a first polymeric nanoparticle has at least one agent covalently bonded to the polymer of the first polymeric nanoparticle and where the composition can also include a second polymeric nanoparticle encapsulating an agent. The agent covalently bonded to the polymer of the polymeric nanoparticle and the agent encapsulated can be the same or different types of agents.

In an aspect, the one or more types of agents can be a polyphenol (e.g., quercetin, resveratrol, and rhamnazin (3′,7-dimethylquercetin, DMQ), derivatives of each of these, salts of each of these, etc.), where at least one agent is covalently bonded to a polymer in a polymer nanoparticle and optionally, an agent is encapsulated in a polymeric nanoparticle (the same or different polymeric nanoparticle). The polymeric nanoparticle has the characteristic that the one or more types of agents can have a sustained release profile (e.g., 1 to 10 days), in particular as compared to agents physically encapsulated. In addition, the present disclosure provides for the synthesis that includes a two-step acylation reaction to covalently bond the agent to the polymer chain and then a step to form the polymer nanoparticles.

The delivery of bioactives to vascular cells requires a system that can target the vascular tissue and release a therapeutic agent for clinical effect. Currently available DCB catheter balloons use mainly hydrophilic excipients such as urea, citrate or iopromide for facilitating transfer within the tissue but do little to facilitate adhesion and maximal transfer. To achieve adequate deposition and delivery of bioactives, disclosed herein are novel polymeric nanoparticle (also referred to as “pNP” in some instances) compositions comprising one or more agents (e.g., therapeutic agent). The agent can be a polyphenol. In an aspect, the agent can be an anti-proliferative therapeutic agents that inhibit restenosis of the vessel and also facilitate re-endothelization of the vessel wall, e.g., resveratrol or derivative thereof and/or quercetin or derivative thereof, covalently bonded to a polymer matrix of a polymeric nanoparticle. The agent being covalently bonded to the polymer can have a sustained or delayed release profile of greater than 1 day, greater than 2 days and up to 6 or 10 days, depending upon the design of the chemical composition and the design of the polymeric nanoparticle. In addition, an agent can be encapsulated within the polymer of the polymeric nanoparticles including the covalently bonded agent or the composition can include two types of polymeric nanoparticles (one with the covalently bonded agent and another that encapsulates the agent).

In one aspect, the disclosure relates to compositions (also referred to as “polymeric nanoparticle compositions (pNP)”), as well as therapeutic composition, having the agents covalently bonded to the polymer nanoparticle for sustained release over the course of days. The disclosed pNP compositions comprising one or more therapeutic agents (e.g., in the same or different polymeric nanoparticles) can be used with devices comprising a drug-coated balloon comprising, as well as used in methods for treating peripheral artery disease using the disclosed compositions and devices.

In an aspect, the agent can be a therapeutic agent selected from the group consisting of resveratrol, pharmaceutically acceptable salts, and pharmaceutically acceptable derivatives thereof; quercetin, pharmaceutically acceptable salts, and pharmaceutically acceptable derivatives thereof; rhamnazin (3′,7-dimethylquercetin, DMQ) pharmaceutically acceptable salts, and pharmaceutically acceptable derivatives thereof; or any combination thereof. Any of these can be covalently bonded to the polymer of the polymeric nanoparticle or encapsulated by the polymer of the polymeric nanoparticle.

In one embodiment, one type of agent can be used (e.g., resveratrol or quercetin) and in another aspect two types of agents (e.g., resveratrol and quercetin) can be used. When two agents are used, they can be within the same polymeric nanoparticle (e.g., one covalently bonded and the other encapsulated) or in different polymeric nanoparticles (e.g., one covalently bonded in one polymeric nanoparticle and another encapsulated in another polymeric nanoparticle). When two agents are used in various embodiments, the ratio of therapeutic agents is in the range selected from the group consisting of about 1:5, about 1:2, about 1:1, about 5:1, or about 2:1 resveratrol to quercetin by weight percent, for example, where resveratrol and quercetin can be replaced with other agents.

In various aspects, the polymer that is covalently bonded to the agent can be a polymer selected from the group consisting of poly(lactide), poly(glycolide), poly(lactide-co-glycolide), poly(caprolactone), poly(lactide-co-caprolactone), poly(glycolide-co-caprolactone), and poly(D,L-lactide-co-glycolide-co-ε-caprolactone). In a still further aspect, the polymer is an A-B block copolymers, wherein block A can be a poly(lactide), a poly(glycolide), or a poly(caprolactone), and wherein block B be independently a polymer distinct from block A and selected from a poly(lactide), a poly(glycolide), or a poly(caprolactone). In a yet further aspect, the polymer is a terpolymer. The terpolymer may be an alternating, random alternating or purely random copolymer or a block copolymer. These polymers can also be used to encapsulate the agent in various embodiments described herein.

In a further aspect, the polymer for each of the embodiments described herein can be a copolymer of lactide and glycolide, i.e., a poly(lactide-co-glycolide), abbreviated as PLGA. In a still further aspect, the polymer is a PLGA with a molar ratio of lactide to glycolide in the PLGA can be 90:10 to 10:90. In a yet further aspect, the polymer is a PLGA with a molar ratio of lactide to glycolide of 75:25 to 25:75. In an even further aspect, the polymer is a PLGA with a molar ratio of lactide to glycolide of 60:40 to 40:60. In some aspects, the polymer is a PLGA with a molar ratio of lactide to glycolide of 50:50. An exemplary PLGA is commercially available under the tradename of Resomer® such as Resomer® RG504H poly(lactic-co-glycolic acid) PLGA 50:50.

In a further aspect, the polymer is a PLGA with a molecular weight of about 5,000 to about 100,000 Dalton. In a still further aspect, the polymer is a PLGA with a molecular weight of about 30,000 to about 60,000 Dalton. In an even further aspect, the polymer is a PLGA with a molecular weight of about 35,000 to about 57,000 Dalton. In a still further aspect, the polymer is a PLGA with a molecular weight of about 38,000 to about 54,000 Dalton. As used herein, the term “molecular weight” refers to “weight average molecular weight.”

The disclosed pNP can be formed by various techniques suitable for forming nanoparticles comprising the disclosed polymer, e.g., an emulsion evaporation technique such as that provided in Example 1.

In various aspects, the disclosed polymeric nanoparticles are about 25 nm to about 500 nanometers in diameter, about 50 nm to about 500 nanometers in diameter, about 50 nm to about 400 nanometers in diameter, about 50 nm to about 300 nanometers in diameter, about 50 nm to about 200 nanometers in diameter, about 50 nm to about 100 nanometers in diameter. In a yet further aspect, the disclosed nanoparticles have a diameter of about 25, 50 nm, 100 nm, 120 nm, 130 nm, 150 nm, 160 nm, 170 nm, 180 nm, 190 nm, 200 nm, 210 nm, 220 nm, 230 nm, 240 nm, 250 nm, any combination of the foregoing values, or any range encompassed by the foregoing values.

In various aspects, the disclosed polymeric nanoparticles can have a positive zeta potential at physiological pH. In a further aspect, the disclosed polymeric nanoparticles can have a zeta potential (expressed in mV) at physiological pH of about 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, any combination of the foregoing values, or any range encompassed by the foregoing values.

In various aspects, the disclosed polymeric nanoparticles can have a pH-independent positive zeta potential. In a further aspect, the disclosed polymeric nanoparticles have a pH-independent zeta potential (expressed in mV of about 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, any combination of the foregoing values, or any range encompassed by the foregoing values.

In various aspects, the present disclosure relates to the administration of resveratrol or derivatives thereof to a subject in order to prevent restenosis and/or the progression or recurrence of coronary heart disease.

Resveratrol may be administered in natural form, i.e., as isolated from grape skins, wine or other plant-derived compositions, or it may be administered as chemically synthesized in the laboratory (e.g., using the methods of Moreno-Manas et al., (1985) Anal. Quim 81:157-61; Jeandet et al., (1991) Am. J. Enol. Vitic. 42:41-46; or Goldberg et al. (1994) Anal. Chem. 66:3959-63), or as obtained commercially, e.g., from the Sigma-Aldrich Corporation (St. Louis, Mo.).

The resveratrol active agent may be administered in the form of a pharmacologically acceptable salt, ester, amide, prodrug or analog, or as a combination thereof. However, conversion of an inactive ester, amide, prodrug or analog to an active form must occur prior to or upon reaching the target tissue or cell. Salts, esters, amides, prodrugs and analogs of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, basic addition salts are prepared from the neutral drug using conventional means, involving reaction of one or more of the active agent's free hydroxyl groups with a suitable base. Generally, the neutral form of the drug is dissolved in a polar organic solvent such as methanol or ethanol and the base is added thereto. The resulting salt either precipitates or may be brought out of solution by addition of a less polar solvent. Suitable bases for forming basic addition salts include, but are not limited to, inorganic bases such as sodium hydroxide, potassium hydroxide, ammonium hydroxide, calcium hydroxide, trimethylamine, or the like. Preparation of esters involves functionalization of hydroxyl groups which may be present within the molecular structure of the drug. The esters are typically acyl-substituted derivatives of free alcohol groups, i.e., moieties which are derived from carboxylic acids of the formula RCOOH where R is alkyl, and preferably is lower alkyl. Esters can be reconverted to the free acids, if desired, by using conventional hydrogenolysis or hydrolysis procedures. Preparation of amides and prodrugs can be carried out in an analogous manner. Other derivatives and analogs of the active agents may be prepared using standard techniques known to those skilled in the art of synthetic organic chemistry, or may be deduced by reference to the pertinent literature (see U.S. Pat. No. 6,022,901).

Resveratrol is known chemically as 3,4′,5-Trihydroxy-trans-stilbene, 5-[(1E)-2-(4-Hydroxyphenyl)ethenyl]-1,3-benzenediol. It has the structure given in Formula 1.

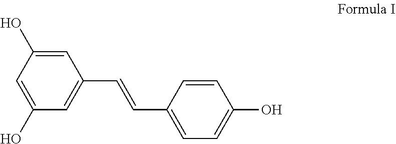 [SMILES: Oc1ccc(/C=C/c2cc(O)cc(O)c2)cc1]

Non-limiting examples of derivatives of cis- and trans-resveratrol are those in which the hydrogen of one or more of the compounds' hydroxyl groups is replaced to form esters or ethers (for example, see Formula I). Ether formation examples include, but are not limited to, the addition of alkyl chains such as methyl and ethyl groups, as well as conjugated mono- or disaccharides such as glucose, galactose, maltose, lactose and sucrose. Additional modifications at the hydroxyl groups might include glucuronidation or sulfation.

Non-limiting examples of derivatives of cis- and trans-resveratrol are those in which the hydrogen of one or more of the compounds' hydroxyl groups is replaced to form esters or ethers (for example, see Formula I). Ether formation examples include, but are not limited to, the addition of alkyl chains such as methyl and ethyl groups, as well as conjugated mono- or disaccharides such as glucose, galactose, maltose, lactose and sucrose. Additional modifications at the hydroxyl groups might include glucuronidation or sulfation.

Esterification products include, but are not limited to, compounds formed through the addition of amino acid segments such as RGD or KGD or other compounds resulting from the reaction of the resveratrol hydroxyl groups with other carboxylic acids.

Additional derivatives include, but are not limited to, those compounds that result from the oxidative dimerization of or functional group addition to the parent resveratrol compound or to a functionalized resveratrol variant. Examples of these compounds include materials resulting from the addition of hydroxyl, methoxy and ethoxy groups at the 4, 2′, and 3′ positions. Dimerization results from the reaction of the ethane bond of one resveratrol molecule with one of the hydroxyl groups on a second resveratrol molecule resulting in the formation of a fused ring system. Alkylation at the 4, 2′, and 3′ positions creates other derivatives through the addition of groups including, but not limited to, methyl, ethyl, and propyl, as well as the addition of larger carbon chains such as 4-methyl-2-pentene, 4-methyl-3-pentene and isopentadiene.

Additional derivatives include, but are not limited to, compounds that arise from the loss of any of the hydroxyl groups of the parent molecule, addition of hydroxyl groups at alternate positions, and any compound that may arise from the previously mentioned reactions to provide a functionalized variant of the dehydroxylated compound.

Structures of exemplary resveratrol derivatives are shown below in Formulas II-VII. The structure of triacetyl resveratrol (TAR) is given in Formula VII.

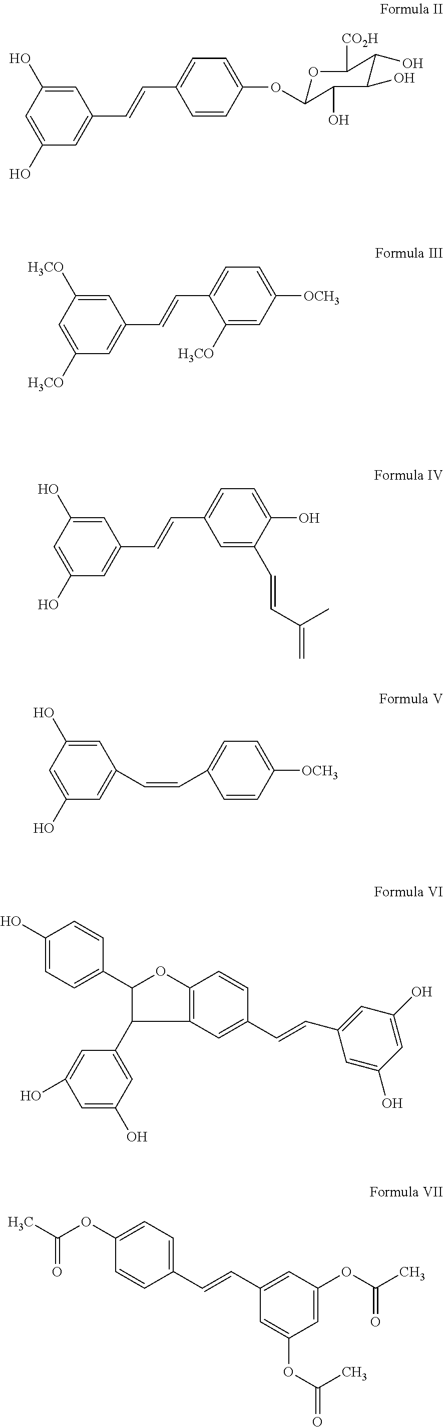

Resveratrol may be involved in many pathways of restenosis. Thus, as used in the disclosed compositions, devices, and methods, resveratrol may address many if not all targets causing a problem from restenosis. For instance, it provides anti-inflammatory benefits and promotes endothelial cell function. Reports have shown that resveratrol stimulates the growth of endothelial progenitor cells, both in vivo and in vitro (see J. Gu, et al., 2006, J Cardiovasc Pharmacol., 47(5): 711-721). Without wishing to be bound by a particular theory, it is possible that this may be a key step in re-endothelialization.

Resveratrol also increases endothelial nitric oxide synthase activity (see Wallerath T et al., Circulation. 2002 Sep. 24; 106(13):1652-8.) Further, resveratrol enhances endothelium-dependent vasorelaxation (Rush J W, Quadrilatero J, Levy A S, Ford R J. Exp Biol Med (Maywood). 2007 June; 232(6):814-22). Therefore, utilizing resveratrol in a DCB and/or other medical device according to the present disclosure provides a multi-faceted approach to reducing restenosis and improving blood flow after stent implantation.

For more information on the use of resveratrol in the treatment of restenosis through methods other than drug-eluting stents, see U.S. Pat. No. 6,022,901, to David William Goodman, titled “Administration of resveratrol to prevent or treat restenosis following coronary intervention”, which is herein incorporated by reference in its entirety.

Resveratrol is a polyphenol that has been linked to the reported cardioprotection of red wine consumption. The reported cardioprotective effects of red wine consumption was prompted by epidemiological studies documenting the “French Paradox,” a term coined to describe the reduced incidence of death due to CHD in areas of southwest France. Inhabitants of this area exhibit increased serum cholesterol and blood pressure and eat more lard and butter than do Americans, yet suffer 40% fewer deaths due to CHD than other western societies. This paradoxical effect is attributed to their daily consumption of red wine. While epidemiological studies suggest a decreased risk of CHD in populations regularly consuming alcohol, considerable data indicate that wine provides greater protection as compared to other alcoholic beverages.

Resveratrol is a phytoalexin polyphenol found in foods such as grapes, mulberries, peanuts, and grapevine. Within the grape itself, resveratrol is most abundant in the skin (ca. 50-100 μg/gm. One fluid ounce of red wine provides ˜160 μg resveratrol. The rapid conjugation of resveratrol to form glucuronides and sulfates has been argued as evidence that orally administered resveratrol concentrations cannot approach therapeutically useful levels. However, immediately after consumption, resveratrol can be measured in the plasma, heart, liver, and kidney. Chronic consumption further increases levels of resveratrol in tissues such as the heart and liver.

Prior reports by other laboratories have indicated that resveratrol acts through a variety of mechanisms to promote vascular health. As an antioxidant polyphenol, it limits the oxidation of low-density lipoprotein, thus inhibiting fatty streak formation. It furthermore exhibits anti-inflammatory effects through an inhibition of NFκB activation. Several labs have demonstrated that resveratrol promotes endothelial function by increasing eNOS activity, and a recent report suggests that the mechanism for this effect is an increase in eNOS phosphorylation. Resveratrol also promotes endothelial protection against oxidant injury, likely via an inhibition of the activation of NADPH oxidase. Finally, resveratrol inhibits adhesion of inflammatory cells to the vascular endothelium by inhibiting the expression of adhesion molecules.

Prior reports demonstrate resveratrol's efficacy in inhibiting proliferation of vascular smooth muscle cells (VSMC). For example, in VSMC stimulated with the mitogens endothelin-1 and platelet-derived growth factor, resveratrol inhibited cell cycle traverse, and in coronary artery smooth muscle, resveratrol inhibited endothelin-1-induced map kinase stimulation.

The mechanisms for these effects are due in part to a resveratrol-mediated ER activation that culminates in an upregulation of tetrahydrobiopterin (BH4) biosynthesis. The inventors have demonstrated that the resulting increase in levels of BH4, a known NOS cofactor, promoted an elevation in NO concentration that culminated in cell cycle arrest. Effects on NO concentration are dependent upon an increase in inducible nitric oxide synthase (iNOS) activity, but not its expression. In addition to this novel ER-dependent pathway, the current invention also shows that resveratrol inhibits NFκB activation very potently.

Thus, according to the present disclosure, resveratrol exerts pleiotropic effects on VSMC proliferation, enhancing NO production through an ER-dependent pathway, but also inhibits NFκB activation through an ER-independent pathway. It is the cooperativity between these two pathways that accounts for the observed effects on VSMC proliferation.

Resveratrol has same binding site as estradiol and behaves as an ER-alpha agonist, however, it has a lower binding affinity than estradiol. This provides protection against estrogenic side effects, such as alternation of the female menstrual cycle and feminization side-effects in males.

In various aspects, the present disclosure relates to the administration of quercetin or derivatives thereof to a subject in order to prevent restenosis and/or the progression or recurrence of coronary heart disease.

Quercetin is typically found in plants as glycone or carbohydrate conjugates. Quercetin itself is an aglycone or aglucon. That is, quercetin does not possess a carbohydrate moiety in its structure. Analogs of quercetin include its glycone conjugates include rutin and thujin. Rutin is also known as quercetin-3-rutinoside. Thujin is also known as quercitrin, quercetin-3-L-rhamnoside, and 3-rhamnosylquercetin. Onions contain conjugates of quercetin and the carbohydrate isorhamnetin, including quercetin-3,4′-di-O-beta glucoside, isorhamnetin-4′-O-beta-glucoside and quercetin-4′-O-beta-glucoside. Quercetin itself is practically insoluble in water. The quercetin carbohydrate conjugates have much greater water solubility then quercetin.

Quercetin is known chemically as 2-(3,4-dihydroxyphenyl)-3,5,7-trihydroxy-4H-1-benzopyran-4-one and 3,3′,4′5,7-pentahydroxy flavone. It has the structure given in Formula VIII.

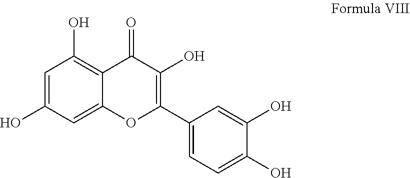 [SMILES: O=c1c(O)c(-c2ccc(O)c(O)c2)oc2cc(O)cc(O)c12]

Quercetin is a phenolic antioxidant and has been shown to inhibit lipid peroxidation. In vitro and animal studies have shown that quercetin inhibits degranulation of mast cells, basophils and neutrophils. Such activities account, in part, for quercetin's anti-inflammatory and immunomodulating activities. Other in vitro and animal studies show that quercetin inhibits tyrosine kinase and reduces the activation of the inflammatory mediator, NF-κB. Further activities of quercetin include anti-viral and anti-cancer activity. Quercetin is further known to inhibit aldose reductase. A quercetin or an analog thereof for use in the present invention can be an inhibitor of tyrosine kinases. The most important biologic activities of quercetin are its inhibition of platelet activation plus its anti-inflammatory properties, as the interaction of these two effects can reduce the incidence of thrombogenesis associated with current generation DES. Quercetin inhibits both platelet activation and platelet aggregation. It enhances platelet-derived nitric oxide to inhibit the activation of a protein kinase C-dependent NADPH oxidase. In addition, quercetin inhibits platelet aggregation through its inhibition of phosphoinositide kinase. Further properties of quercetin or its analogs that are relevant in the context of the present invention include: inhibition of cell cycle, inhibition of smooth muscle cell proliferation and/or migration. Suitable analogs/derivatives of quercetin include its glycone conjugates rutin and thujin (See U.S. Patent Application Publication No. 2007/0212386 (Patravale et al.)).

Quercetin and/or its analogs may be capable of exerting the above activities when used singly. However, the above properties of quercetin and/or its analogs may be further enhanced by exploiting the synergy between quercetin and/or its analogs and further therapeutic agents (as disclosed herein), such as resveratrol and/or its derivatives.

In one embodiment, the combination of polymer and pharmaceutically active agent comprise a combination of pharmaceutically active agents. If more than one pharmaceutically active agent is used, they can be present in combination in the same layer, or in separate polymer layers. Exemplary combinations include resveratrol plus quercetin separately or in combination in one or more coatings and resveratrol or quercetin alone or in combination in one or more coatings.

Exemplary derivatives of quercetin are those in which the hydrogen of one or more of the compounds' hydroxyl groups, most commonly the 3 hydroxyl is replaced to form esters or ethers (see for example Formula VIII). Ether formation examples include, but are not limited to, the addition of alkyl chains such as methyl and ethyl groups, as well as deoxy sugars such as fucose and rhamnose. Esterification products include, but are not limited to; compounds formed through the reaction of carboxylic acid containing materials such as acetic acid, propionic acid and palmitic acid. Urethane derivatives of quercetin include, but are not limited to; amino acid ester carbamates formed by the addition of materials such as benzyl 2-isocyanatoacetate and (S)-methyl 2-isocyanatopropanoate.

Additional quercetin derivatives include, but are not limited to, compounds that can be described as metabolites formed by the addition of sugar-like derivatives such as glucuronyl groups at any of the hydroxyl positions. Examples of these metabolites include 7-O-glucuronyl-quercetin and 3′-O-glucuronyl-quercetin.

Additional derivatives include, but are not limited to, compounds that arise from the loss of any of the hydroxyl groups of the parent molecule, addition of hydroxyl groups at alternate positions, and any compound that may arise from the previously mentioned reactions to provide a functionalized variant of the dehydroxylated compound.

Quercetin is also a polyphenol present in red wine and it is likewise reported to exert protection against atherosclerosis. From a pharmacological point of view, an exemplary drug combination of the present invention, resveratrol and quercetin, appears reasonable, red wine is actually a combination of low levels of many bioactive polyphenols that act synergistically to exert the effects observed clinically for chronic red wine consumption.

Quercetin is an inhibitor of both platelet and NFκB activation. The addition of quercetin to the DES of the present invention should potentiate the effects of resveratrol on VSMC proliferation by boosting the inhibitory effects on NFκB activation. Further, strong inhibition NFκB should also potentiate resveratrol-mediated inhibition of the inflammatory component of restenosis. Addition of quercetin should also limit platelet activation, which is a part of the inflammatory response to balloon angioplasty and stent implantation that leads to restenosis. Alternatively, another agent or agents which inhibit platelet activation and/or aggregation could be utilized in place of quercetin with resveratrol. Such alternative options include, but are not limited to, aspirin, ticlopidine, clopidogrel, dipyridamole, and the like.

In some aspects, the quercetin used is as methylated quercetin. Without wishing to be bound by a particular theory, it is possible that methylated quercetin can permit maintenance of quercetin drug levels at a tissue site with a time course similar to resveratrol or methylated resveratrol.

Structures of exemplary quercetin derivatives are shown below in Formulas IX-XII.

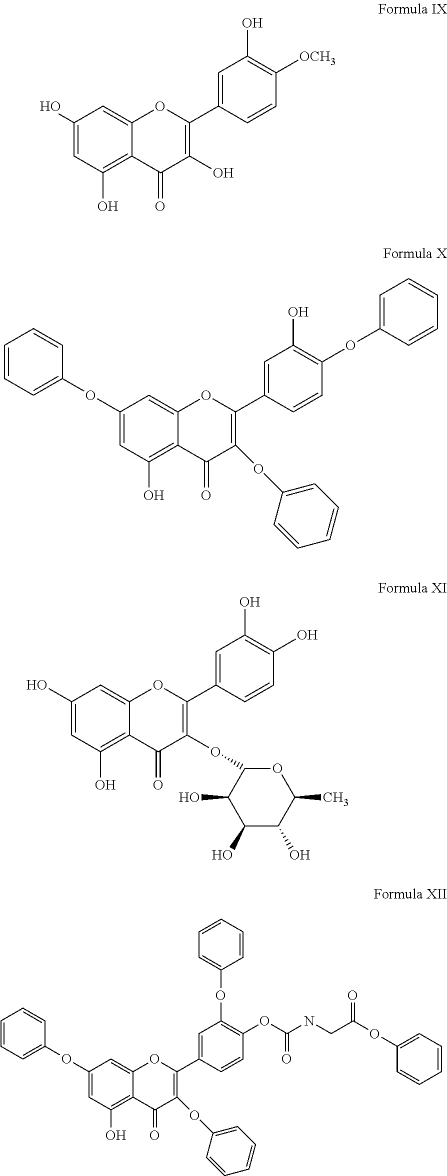

In aspects, the composition, as well as therapeutic compositions, can include one or more therapeutic agents as described herein. In various aspects, the disclosed therapeutic agent comprises a first therapeutic agent selected from the group consisting of resveratrol, pharmaceutically acceptable salts, and pharmaceutically acceptable derivatives thereof and optionally a second therapeutic agent selected from the group consisting of quercetin, pharmaceutically acceptable salts, and pharmaceutically acceptable derivatives thereof. In an embodiment, both the first therapeutic agent and the second therapeutic agent can be covalently bonded to the polymer of the polymeric nanoparticle. In another embodiment, one of the first therapeutic agent and the second therapeutic agent is covalently bonded to the polymer of the polymeric nanoparticle while the other is encapsulated in the polymer of the same polymeric nanoparticle or a different polymeric nanoparticle.

In a further aspect, the first therapeutic agent can be used in combination with a lower amount of the second therapeutic agent (e.g., same polymeric nanoparticle or in the same composition in different polymeric nanoparticles). In a still further aspect, the first therapeutic agent is present in a ratio to the second therapeutic agent of from about 1:1 to about 5:1. In a yet further aspect, the first therapeutic agent is present in a ratio to the second therapeutic agent of from about 1:1 to about 2.5:1. In an even further aspect, the first therapeutic agent is present in a ratio to the second therapeutic agent of from about 1.5:1 to about 2.5:1.

In various aspects, the first therapeutic agent can be used in combination with a lower amount of the second therapeutic agent such that the first therapeutic agent is present in a ratio to the second therapeutic agent of 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1; any combination of the foregoing values; or any range encompassed by two of the foregoing values.

In various aspects, the first therapeutic agent can be used in combination with a lower amount of the second therapeutic agent such that the first therapeutic agent is present in a ratio to the second therapeutic agent of 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.32, 2.33, 2.34, 2.35, 2.36, 2.37, 2.38, 2.39, 2.40, 2.41, 2.42, 2.43, 2.44, 2.45, 2.46, 2.47, 2.48, 2.49, 2.50; any combination of the foregoing values; or any range encompassed by two of the foregoing values.

The present disclosure provides for drug coating devices such as balloon catheters, vascular grafts, self-expandable stents, balloon-expandable stents, and stent-grafts and the like. In general, embodiments of the composition as provided herein can be disposed on a surface of the device so that the agents in the composition can act as intended. While the following describes details specific to a catheter balloon, this in not intended to limit the device to a catheter balloon and other devices can include compositions of the present disclosure.

The present disclosure also provides a DCB catheter balloon comprises a pNP coating comprising one or more therapeutic agents covalently bonded to the polymer of the polymeric nanoparticle, with optionally one or more therapeutic agents encapsulated by the polymer of the polymeric nanoparticle or the pNP coating includes two types of polymeric nanoparticles, one having at least on therapeutic agent covalently attached to the polymer of the polymeric nanoparticle and another polymeric nanoparticle encapsulating at least one type of therapeutic agent. In some aspects, the composition including the polymeric nanoparticle(s) is applied directly on the outer surface of a DCB catheter balloon.

In a further aspect, the present disclosure pertains to an implantable medical device, comprising: an expandable balloon catheter having an outer surface; and an adherent layer on the balloon catheter comprising a pNP comprising a therapeutic agent selected from the group consisting of resveratrol, pharmaceutically acceptable salts, and pharmaceutically acceptable derivatives thereof, where at least one therapeutic agent is covalently bonded to the polymer nanoparticle and optionally another therapeutic agent is encapsulated by the same or a different polymeric nanoparticle.

In a further aspect, the present disclosure pertains to an implantable medical device, comprising: an expandable balloon catheter having an outer surface; and an adherent layer on the balloon catheter comprising a pNP comprising a therapeutic agent selected from the group consisting of quercetin, pharmaceutically acceptable salts, and pharmaceutically acceptable derivatives thereof, where at least one therapeutic agent is covalently bonded to the polymer nanoparticle and optionally another therapeutic agent is encapsulated by the same or a different polymeric nanoparticle.

In a further aspect, the present disclosure pertains to an implantable medical device, comprising: an expandable balloon catheter having an outer surface; and an adherent layer on the balloon catheter comprising a pNP comprising a first therapeutic agent selected from the group consisting of resveratrol, pharmaceutically acceptable salts, and pharmaceutically acceptable derivatives thereof, and an optional second therapeutic agent selected from the group consisting of quercetin, pharmaceutically acceptable salts, and pharmaceutically acceptable derivatives thereof, where at least one therapeutic agent is covalently bonded to the polymer nanoparticle and optionally another therapeutic agent is encapsulated by the same or a different polymeric nanoparticle.

In a further aspect, the present disclosure pertains to an implantable medical device, comprising: an expandable balloon catheter having an outer surface; and an adherent layer on the balloon catheter comprising a pNP comprising a first therapeutic agent selected from the group consisting of resveratrol, pharmaceutically acceptable salts, and pharmaceutically acceptable derivatives thereof, and a second therapeutic agent selected from the group consisting of quercetin, pharmaceutically acceptable salts, and pharmaceutically acceptable derivatives thereof, where at least one therapeutic agent is covalently bonded to the polymer nanoparticle and optionally another therapeutic agent is encapsulated by the same or a different polymeric nanoparticle.

In various aspects, the DCB catheter balloon made of a suitable medical grade polymer with a suitable combination of strength, flexibility and friction characteristics. In some aspects, the balloon comprises a nylon. In a further aspect, the balloon comprises a polyamide block copolymer obtained by polycondensation of a carboxylic acid polyamide (PA6, PA11, PA12) with an alcohol termination polyether (Polytetramethylene glycol PTMG), PEG). Exemplary nylons include those available under the tradenames PEBAX® (Arkema) and VESTAMID® E (Evonik Industries). In a further aspect, the DCB catheter balloon comprises one or more polymers possessing a negative zeta potential, e.g., a nylon block copolymer such as a PEBAX®. In alternative aspects, the DCB catheter balloon comprises one or more of a polyethylene, polyurethane, polypropylene and similar materials.

In various aspects, the balloon can be smooth-walled, or in some aspects provided with grooves or with pores, provided in part increase the surface area of the balloon portion.

The DCB catheter balloon is typically expandable up to a predetermined size and should preferably be pressure resistant in order to be able expand stenotic arteries back to their original diameter.

In an aspect, the pNP coating is a composition or a therapeutic composition. The pNP coating comprises a pNP with one or more therapeutic agents covalently bonded to the polymer of the polymeric nanoparticle and/or one or more therapeutic agents encapsulated by the same or different polymeric nanoparticles. Examples of the one or more therapeutic agents which can be covalently bonded to the polymer of the polymeric nanoparticle or encapsulated, which is then included in the pNP coating on the DCB catheter, include but are not limited to, resveratrol and quercetin for use in/on a coating on a balloon catheter.

Dip coating techniques can be used for coating the surface of a balloon although other methods may also be employed such as spray coating (e.g., ultrasonic spray coating) or electrospray coating methods. In a further aspect, an electrospray method is used to provide the pNP coating, the first coating, and/or the second coating. Coating is typically comprised of a single layer but may also comprise multiple layers depending on the content and release profile of drug contained in the coating.

In various aspects, the present disclosure pertains to a drug coated balloon catheter, comprising: an expandable balloon having an outer surface; and a nanoparticle coating the outer surface of expandable balloon comprising a disclosed pNP composition such that a concentration of the agent based on a surface area of the balloon ranges from about 1 to about 5 μg/mm².

In a further aspect, the nanoparticle coating the outer surface of the expandable balloon comprises a disclosed pNP composition at a concentration of the agent, in units of μg/mm², is about 1.0, 1.1, 1.2, 1.3, 1.4, 1.5, 1.6., 1.7, 1.8, 1.9, 2.0, 2.1, 2.2, 2.3, 2.4, 2.5, 2.6., 2.7, 2.8, 2.9, 3.0, 3.1, 3.2, 3.3, 3.4, 3.5, 3.6., 3.7, 3.8, 3.9, 4.0, 4.1, 4.2, 4.3, 4.4, 4.5, 4.6., 4.7, 4.8, 4.9, 5.0; any combination of the foregoing values; or a range encompassed by any two of the foregoing values.

The disclosed DCB catheter balloons are useful in revascularization, catheterization, balloon expansion and stent delivery procedures and methods described herein. In a stent delivery procedure for example, a drug coated balloon according to the present disclosure may also incidentally deliver drugs to vessel areas that are not situated at the localized situs of implant of a stent. Such incidental delivery of drug from the surface of the balloon is of particular utility for small and tortuous vessel passages leading up to the site of interest.

Furthermore, healing and re-endothelialization of stent struts that do not carry antiproliferative agents can be facilitated by the use of drug coated balloons.

With a DCB catheter, the balloon walls contact the vessel walls when inflated, and the drug is released some initially but is released over a sustained time from about 1-10 days and about 1 to 6 day.

The present disclosure also pertains to methods of treating a vascular disease comprising treating a subject with a device such as those provided herein. In a further aspect, the disclosed methods are methods for treating at least one disease or condition associated with vascular injury or angioplasty. Angioplasty may be performed as part of “revascularization” treatment for “artherosclerosis,” which as used herein means diseases in which plaque, made up of cholesterol, fats, calcium, and scar tissue, builds up in the wall of blood vessels, narrowing the lumen and interfering with blood flow. “Revascularization,” as used herein means any treatment that re-establishes brisk blood flow through a narrowed artery, including bypass surgery, angioplasty, stenting, and other interventional procedures. Secondary complications following revascularization may include restenosis, neointima, neointimal hyperplasia and thrombosis. “Restenosis,” as used herein is defined as the re-narrowing of an artery in the same location of a previous treatment; clinical restenosis is the manifestation of an ischemic event, usually in the form of recurrent angina. “Neointima,” as used herein is defined as the scar tissue made up of cells and cell secretions that often forms as a result of vessel injury following angioplasty or stent placement as part of the natural healing process. “Neointimal hyperplasia,” as used herein means excessive growth of smooth muscle cells from the inner lining of the artery. After angioplasty and/or stenting, excessive growth of these cells can narrow the artery again. “Thrombosis,” as used herein means the formation of a blood clot within a blood vessel or the heart cavity itself and a “thrombus” is a blood clot. The present disclosure also includes treating vascular disease in a subject, where the vascular disease includes vascular calcification.

Three pathophysiological phases can be distinguished subsequent to revascularization. Stage I, the thrombotic phase (days 0-3 after revascularization). This stage consists of rapid thrombus formation. The initial response to arterial injury is explosive activation, adhesion, aggregation, and platelet deposition. The platelet thrombus may frequently be large and can grow large enough to occlude the vessel, as occurs in myocardial infarction. Within 24 hours, fibrin-rich thrombus accumulates around the platelet site. Two morphologic features are prominent: 1) platelet/fibrin, and 2) fibrin/red cell thrombus. The platelets are densely clumped at the injury site, with the fibrin/red cell thrombus attached to the platelet mass. Stage II, the recruitment phase (days 3-8). The thrombus at arterial injury sites develops an endothelial cell layer. Shortly after the endothelial cells appear, an intense cellular infiltration occurs. The infiltration is principally monocytes that become macrophages as they leave the bloodstream and migrate into the subendothelial mural thrombus. Lymphocytes also are present, and both types of cells demarginate from the bloodstream. This infiltrate develops from the luminal side of the injured artery, and the cells migrate progressively deeper into the mural thrombus. Stage Ill, the proliferative phase: (day 8 to final healing). Actin-positive cells colonize the residual thrombus from the lumen, forming a “cap” across the top of the mural thrombus in this final stage. The cells progressively proliferate toward the injured media, resorbing thrombus until it is completely gone and replaced by neointimal cells. At this time the healing is complete. In the pig this process requires 21-40 days, depending on residual thrombus thickness. Smooth muscle cell migration and proliferation into the degenerated thrombus increases neointimal volume, appearing greater than that of thrombus alone. The smooth muscle cells migrate from sites distant to the injury location, and the resorbing thrombus becomes a bioabsorbable “proliferation matrix” for neointimal cells to migrate and replicate. The thrombus is colonized at progressively deeper levels until neointimal healing is complete.

In a further aspect, the disclosed methods can be used to treat these conditions subsequent to revascularization, such as those conditions subsequent to any of the three stages described above, e.g., activation, adhesion, aggregation, platelet deposition, thrombosis, platelet aggregation, proliferation, and neointima.

In a further aspect, the therapeutic agent is for the prevention or treatment of restenosis subsequent to angioplasty, such as the inhibition of neointimal hyperplasia subsequent to angioplasty.

In a further aspect, the disclosed methods are directed to the prevention of acute, subacute and chronic secondary complications associated with angioplasty. Such secondary complications subsequent to and/or associated with angioplasty are defined herein above and include, e.g., restenosis, neointima, neointimal hyperplasia, thrombosis and inflammation.

In a further aspect, the disclosed methods are directed to treating undesired cell proliferation, which is often a component of many disease processes. Undesired cell growth can be a component of restenosis, the recurrence of stenosis or artery stricture after corrective surgery. Restenosis occurs after coronary artery bypass (CAB), endarterectomy, heart transplantation, or after angioplasty, atherectomy, laser ablation or stenting. Restenosis is the result of injury to the blood vessel wall during the lumen opening procedure. In some patients, the injury initiates a repair response that is characterized by smooth muscle cell proliferation referred to as “hyperplasia” in the region traumatized by the angioplasty. This proliferation of smooth muscle cells re-narrows the lumen that was opened by the angioplasty within a few weeks to a few months, thereby necessitating a repeat angioplasty or other procedure to alleviate the restenosis.

In a further aspect, the disclosed methods provide for delivery of the disclosed therapeutic agents locally to reduce side effects from high dose systemic delivery.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

### Example 1

Peripheral artery disease (PAD) is a systemic atherosclerotic disease that affects approximately 202 million people worldwide. With over 8 million diagnoses, PAD is one of the most common causes of mortality in the United States (1-4). Moreover, atherosclerotic diseases like PAD are becoming a world-wide problem (5). PAD is characterized by debilitating atherosclerotic occlusion of arteries in the lower extremities, resulting in an obstruction of blood flow (1, 6). Though a disease of the extremities, left untreated, PAD can culminate in catastrophic consequences like stroke, myocardial infarction, and death (2, 7). The most common symptom among patients with PAD is intermittent claudication, but it is often asymptomatic, under-diagnosed and under-treated, resulting in a reduced functional capacity and quality of life. In its most severe form, the resulting limb ischemia can necessitate limb amputation (2-4). To treat lower extremity PAD, clinicians often revascularize the affected artery or arteries using an endovascular procedure known as angioplasty, achieved using balloon dilation and sometimes, placement of a stent (8-11), Angioplasty is a technique of mechanically widening a blood vessel that has been narrowed or obstructed due to atherosclerosis (12). In PAD, balloon angioplasty is favored over stenting due to the small diameter of the affected arteries and the preponderance of stent fractures occurring in clinical cases (13, 14). Balloon angioplasty allows slow vessel stretching to enlarge the lumen (12). Unfortunately, it also induces stretch and strain to the vessel wall, and the injury it imparts induces a series of cellular events culminating in the formation of a new lesion (15). Restenosis or vessel re-narrowing after implantation remains a complication of vascular interventions (16). Early restenosis and neointimal hyperplasia within the stented vessels have been attributed to deep vascular injury, with fracture of the internal elastic lamina (17). Intimal hyperplasia includes inflammatory phenomena, migration, and proliferation of smooth muscle cells and also, extracellular matrix deposition (17). These events culminate in a thickened vessel wall that obstructs blood flow (15, 18).

Current protocols for the prevention and therapy of restenosis after angioplasty/stenting are based on sustained, antiproliferative drug release into the vessel wall (19). Drug-coated balloons (DCB) have recently emerged as a treatment for peripheral artery (19-22) and coronary in-stent restenosis (23). The concept of DCB therapy relies on healing of the vessel wall after a rapid release of drug locally but retention of the drug within the vessel wall long enough to impact deleterious cellular events occurring early after the procedure. DCBs require three fundamental elements: a semi-compliant angioplasty balloon, an antiproliferative drug and a drug carrier (23). DCB releasing the chemotherapy agent paclitaxel have been approved by the FDA. Paclitaxel is highly lipophilic and binds quickly and tightly to tissue, which results in rapid cellular uptake and long-term retention at the site of delivery. This treatment comes with major disadvantages such as: systemic toxicity (15, 24), the release of paclitaxel before arrival at the lesion site due to direct application of drug to the balloon surface (15, 24) and delayed re-endothelialization, as demonstrated by animal studies utilizing paclitaxel-eluting stents (25, 26). In addition, recent alerts issued by the FDA identified a late mortality signal in study subjects treated with paclitaxel-coated balloons. The relative risk for increased mortality at 5 years was 1.57 (95% confidence interval 1.16-2.13), which corresponds to a 57% relative increase in mortality in patients treated with paclitaxel-coated devices (27). Therefore, studies focused on controlled delivery of other anti-proliferative agents have evolved. Our own prior research focused on two synergistic polyphenols—resveratrol and quercetin—and these studies demonstrated that the two have low toxicity and reduce vascular smooth muscle proliferation but promote re-endothelialization, both in vitro and in vivo (15, 28). We were also successful in developing a drug-eluting coating that successfully achieves slow release of resveratrol (i.e., over several days), but by comparison, release of quercetin was more rapid and less protracted (15).

Within this framework, the aim of this study was to develop polymeric nanoparticles (pNP) for quercetin delivery that were capable of a high entrapment, slow release of drug and antiproliferative activity. Poly (lactic-co-glycolic acid) (PLGA) nanoparticles with entrapped quercetin (pNP(eQ)), a dimethoxy quercetin (i.e., rhamnazin (3′,7-dimethylquercetin, DMQ), designated pNP(eDMQ)), as well as quercetin conjugated to PLGA (pNP(cQ)), were developed. Using an ultrasonic coating method, miniaturized balloon catheters were coated with pNP, and nanoparticle characteristics, drug loading, drug release, and efficacy in reducing vascular smooth muscle cell proliferation were assessed. With respect to the coated balloons, we also determined particle deposition on the balloon surface, assessed as total pNP and drug loading, as well as coating uniformity. We aimed to achieve uniformly coated balloons, with the particles firmly adhered. Our overarching project goal is to achieve minimal loss of drug from the balloon surface during transit to the lesioned area, but upon inflation within the lesioned artery, the particles transfer and attach firmly to the vessel wall, where the coating begins releasing polyphenols. As such, we aim to achieve a controlled and localized administration of the active substance in the affected area.

A secondary aim of our design was to enable pNP adhesion to calcified lesions. Vascular calcification is a common occurrence in PAD and compared to coronary artery disease, can be extensive (29). The accumulation of calcium and phosphate in the intimal and medial layers of the vessel are typical of patients with PAD, particularly those with chronic kidney disease and diabetes mellitus (30). Calcification is a key contributor to poorer outcomes after angioplasty, as it leads to altered compliance, flow-limiting dissections and acute vessel recoil (31). Moreover, late lumen loss after paclitaxel-coated balloon therapy was shown correlated with circumferential calcification (32), and hypotheses are that such outcomes are due to an inability of the calcified lesion to absorb paclitaxel. Thus, in some experiments, we tested whether our pNP coating was capable of strong adhesion to cells in which calcium accumulation was induced experimentally.

### Materials and Methods

### Materials

The following materials were obtained from Sigma-Aldrich (St. Louis, MO): Resomer RG504H poly (D, L-lactide-co-glycolide), PLGA 50:50 (molecular weight 38,000-54,000), acetone and poly (vinyl alcohol) (PVA 31,000-50,000; 87-89% hydrolysed), quercetin and rhamnazin (3′,7-dimethylquercetin, DMQ). PLGA covalently modified with quercetin was synthetized in the laboratory. Analytical grade chemicals and reagents were used for this study.

Conjugation of PLGA with Quercetin

The coupling of quercetin to PLGA was based on an acylation reaction. The first step was PLGA activation. Briefly, 2 g of PLGA was dissolved in 50 mL DCM at room temperature in a 3-neck round bottom flask. A bubbler bottle with 1 M sodium hydroxide NaOH was required to neutralize HCl produced during the reaction under nitrogen. After complete dissolution of PLGA at room temperature, 10 eq. of oxalyl chloride was added dropwise with a glass syringe, along with 3 mL of DMF. The reaction was performed at room temperature with mild stirring for 5 hours. Next, the solution was concentrated with a Buchi R-300 Rotavapor (Buchi Corporation, New Castle, DE). The activated PLGA polymer was precipitated by addition of 200-300 mL of ethyl ether. The white precipitate was washed at least three times with ethyl ether to remove impurities. The solids were dried overnight under high vacuum. The second reaction was performed by dissolution of 1 g of dry PLGA-CI in 25 mL of DMSO, which was added dropwise to 35 mg of quercetin dissolved in 20 mL of DMSO. The reaction was performed overnight at room temperature under nitrogen. The PLGA-quercetin polymer was precipitated by addition of 150 mL of ethyl ether; the precipitation was repeated three times. The precipitated polymer was suspended in 80 mL of DCM and the organic phase was washed with 200 mL of water to remove unreacted quercetin. The process was repeated to obtain a clear supernatant. Finally, the DCM was evaporated with a Buchi R-300 Rotavapor, and the polymer was dried under high vacuum for 3 days at 30° C. The PLGA-quercetin copolymer was stored at 2-4° C. for further characterization and use in nanoparticle synthesis.

### pNP Synthesis

### Synthesis of PLGA-Eudragit RL-100 Nanoparticles

The polymeric nanoparticles were synthesized employing a single emulsion evaporation technique. Briefly, an organic phase was created by mixing Eudragit RL 100 (60 mg) and PLGA (200 mg) in ethyl acetate to acetone (8:2) solution (6 mL), with mild stirring at room temperature for 30 minutes. Next, quercetin or rhamnazin (3′,7-dimethylquercetin, DMQ) was added to the organic phase. Rhamnazin (3′,7-dimethylquercetin, DMQ) was used to test whether alkylation of quercetin resulted in a protracted release profile. After 15 min and with continued stirring at room temperature, the organic phase was poured dropwise into 60 mL of aqueous phase containing 4 mg/mL Tween 80. To reduce droplet size, the emulsion was microfluidized with an M-110P Microfluidizer (Microfluidics Corp, Westwood, MA) at 4° C., 30,000 PSI, with four passes. Ethyl acetate in the suspension was evaporated using a Buchi R-300 Rotavapor (Buchi Corp., New Castle, DE) under vacuum at 32° C. for 2 h. Finally, the nanoparticle suspension was mixed with trehalose at a 1:2 mass ratio, and the suspension was freeze-dried with a FreeZone 2.5 (Labconco Corp., Kansas City, MO) at 32° C. for 2 days. A 2 mL solution of polyvinyl alcohol (PVA; 30 mg) was added before freeze-drying to minimize aggregation after polymeric nanoparticle resuspension. The powdered samples were kept at −80° C. until further characterization and use. In some studies, PLGA was covalently modified with Q prior to pNP synthesis (see section 2.2), but all other steps were identical. The mean size, PDI and zeta potential of the polymeric nanoparticles were measured by Dynamic Light Scattering (DLS) with a Malvern Zetasizer nano ZS (Malvern Panalytical inc, Westborough, MA). Because pilot studies demonstrated an impact of trehalose on cell growth, for studies examining the effect of nano-delivered quercetin on vascular smooth muscle cell proliferation, the pNP were prepared fresh on the day of the experiment, without freeze-drying and without trehalose. However, all other components were maintained at a similar ratio to ensure that the pNP formulations for the two studies were similar.

### Nanoparticle Characterization

### Morphology

Transmission electron microscopy (TEM) was accomplished using a JEOL JM-1400 (JEOL USA Inc., Peabody, MA) and an accelerating voltage of 120 kV. As such, TEM was used to analyse the structure of empty PLGA polymeric nanoparticles (pNP(E)), PLGA NP with entrapped rhamnazin (3′,7-dimethylquercetin, DMQ) (pNP(eDMQ)), PLGA pNP with entrapped quercetin (pNP(eQ)) and PLGA NP with conjugated quercetin (pNP(cQ)). One drop of the pNP resuspension in nanopure water was placed onto a carbon film 400 mesh copper grid, and the excess amount of solution was removed with sterile filter paper. A solution of 2% uranyl acetate was used for staining. After 5 min, a separate sterile filter paper was utilized to remove excess uranyl acetate.

### Size Distribution and Zeta Potential Characterization

Dynamic light scattering (DLS) (Malvern Panalytical, Westborough, MA) was employed to characterize the nanoparticles for size, polydispersity and zeta potential. After resuspension in low resistivity water, a disposable capillary cell of 1 mL volume was used to measure size, polydispersity index (PDI), and zeta potential (Smoluchowski model) for NP.

### Drug Release and Biologic Efficacy

### Drug Release Protocol

The release profiles were performed by placing 10 mg/mL PLGA-Eudragit RL100 NP (pNP(eQ), pNP(eDMQ), pNP(cQ)) in dialysis membrane (molecular weight cut-off of 12,000/14,000 g/mol, regenerated cellulose, Fisher Scientific). Sterile PBS was used for sample resuspension. The samples were dialyzed against 800 mL of PBS at 37° C. under continuous stirring, and PBS was replaced every 8 h in the first 12 h and then every 24 h. At pre-determined time points, 0.2 mL samples were taken from inside the dialysis bag (nanoparticle solution) and to prevent quercetin oxidation, was mixed with 20 μL of 50 mM ascorbic acid. Finally, 800 μL of DMF was added to extract the active components. The samples were vortexed for 1 h at room temperature and then stored at −80° C. until drug concentrations could be measured using a high-performance liquid chromatography (HPLC) method we described previously (15).

### Cell Proliferation Assay

Rat aortic smooth muscle cells (RAOSMC; Cell Applications, Inc., San Diego, CA) were grown to 50-60% confluency in 24-well plates. After serum-starvation for 72 hours to achieve cell cycle synchronization, the cells were stimulated with phenol red-free medium containing 10% Fetal Bovine Serum (FBS) and 0.4 mg/mL of either empty pNP (pNP(E)), or quercetin-containing pNP, including pNP(eQ), pNP(eDMQ) or pNP(cQ). Cell proliferation was assessed by following the rate of DNA synthesis, determined as the amount of 5-bromo-2′-deoxy-uridine (BrdU) incorporation (Roche BrDU Labeling and Detection Kit II, Sigma-Aldrich, St. Louis, MO). Briefly, 100 μL BrdU labelling reagent was added to each well and the plates were incubated for 2 h at 37° C. The medium was aspirated, 300 μL Fixdenat was added, and the plates were incubated for 30 min at room temperature. Next, the Fixdenat was aspirated and 300 μL peroxidase conjugated anti-BrdU antibody was added to all wells, including the background control wells, and the plates were incubated for 90 min at room temperature. The wells were then washed 3 times with 300 μL washing buffer, and 300 μL of substrate were added and allowed to incubate for 2 minutes in dark conditions and at room temperature. Finally, 75 μL 1M H₂SO₄ were added to each well, and after rotating for 2 minutes, absorbance was read at 450 nm (reference 690 nm) using a Biotek Synergy microplate reader. Data were expressed as a percent of control cells stimulated with only 10% FBS but with no nanoparticles.

### Adhesion Study Protocol

Because endothelial cells are denuded during angioplasty, smooth muscle cells are the predominate cell type exposed to the balloon to accept pNP containing polyphenols during balloon inflation. Moreover, as explained in the introduction, these cells are typically calcium-laden in PAD. Thus, to model advanced calcified lesion in PAD, RAOSMC were cultured and maintained in a black-walled, clear bottom, tissue-culture treated plates with growth medium compared to calcification medium for two weeks (33). Growth medium contained DMEM with 10% fetal calf serum. Calcification medium contained high glucose (4.5 g/L) DMEM with 10% fetal calf serum, 100 U/mL penicillin, 100 μg/mL streptomycin, 6 mmol/L CaCl₂, 10 mmol/L sodium pyruvate, 10-6 mol/L insulin, 50 μg/mL ascorbic acid, 10 mmol/L β-glycerophosphate and 10-7 mol/L dexamethasone. Calcification was confirmed using Von Kossa staining (S1 Fig). Next, 10 mg/mL suspensions of pNP(E), pNP(eQ), and pNP(cQ) were diluted in PBS to a final concentration of 2.0 mg/mL. From each suspension 100 μL were placed in wells of the culture plates containing calcified/uncalcified RAOSMC and the cells were incubated at 37° C. for 2 hours. We selected 2 hours because pilot observations determined that 2 hours was the minimum amount of time required for pNP to fall to the bottom of the well and adhere. Drug-containing medium was then removed and the cells were subjected to a 100 μL PBS wash before every well was aspirated to dryness. Fluorescence intensity was quantified before and after washing using a Biotek Synergy 2 fluorescence plate reader. Measures of fluorescence detected for cells containing no pNP were used for background correction. In addition, after washing, green fluorescence images of wells were captured on a ZOE Fluorescent Cell Imager (BIO-RAD). Lysis buffer was then placed in wells so that protein levels could be quantified by BCA protein assay. Measures of fluorescence units were normalized to μg protein in each well.

### Balloon Coating and Characterization

### Balloon Fabrication and Ultrasonic Coating

A balloon catheter with a 13.8 cm extrusion, a 2.7 FR polycarbonate luer fitting and a 1.25 mm×10 mm PET over-the-wire balloon was custom manufactured by Interplex Medical, LLC (Milford, OH). Eight balloons were shipped directly to Sono-Tek Corporation (Milton, NY), where they were professionally coated with pNP(eQ) using the following ultrasonic coating method. First, the sample for coating was drawn into a 10 mL syringe, was affixed to a MediCoat BCC coating system and was allowed to reach room temperature. Prior to coating, an atomization test was conducted using a Sono-Tek 48 kHz Accumist nozzle. The material was found to coat flawlessly at low power output. A 3-axis XYZ Gantry System (500 mm×500 mm×100 mm), a rotator and the appropriate mounting hardware was interfaced to the system so as to accommodate the balloon catheter. The balloons were inflated and coated using 5, 10, 15 or 20 layers with n=2 balloons coated per group, so that the impact of deposition amount on uniformity and drug loading could be determined. Note that the prohibitive costs of the balloon catheters precluded our ability to test more than 8 balloons.

### Fluorescence Imaging to Quantify pNP Loading and Uniformity of Coating

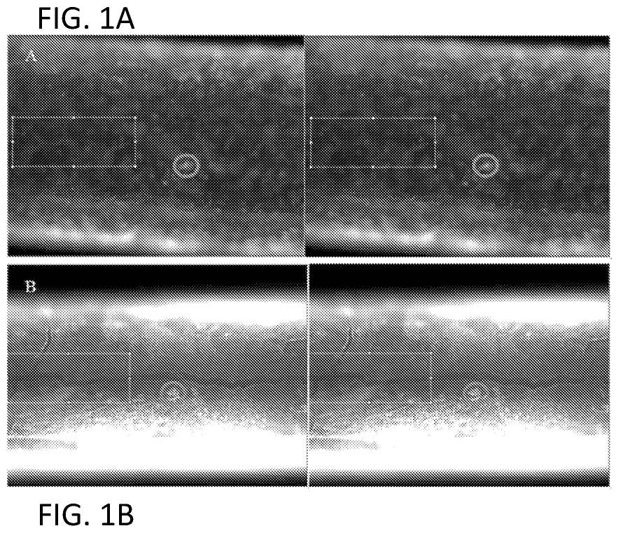

The balloons were affixed on microscopic slides with tape, making every effort to keep them aligned with the center of the slide (deviation of 3°). Microscopic images were acquired at 4× magnification using a Cytation 3 Image reader (BioTek Instruments Inc, Winooski, VT) in TIFF format, with a 16-bit resolution, both in the visible range and in fluorescent mode. The field of view was panned over in sequential images in order to image the whole balloon in a sequence, and 7-9 images were captured for each balloon. In one case the balloon exceeded the image edges at 4× magnification, so a “top” and a “bottom” image were later combined using the Stitching (34) plugin provided by Fiji (formerly ImageJ) analysis software. The images were acquired with the same imaging parameters (LED intensity=3, integration time=100 ms, camera gain=14), preselected based on the “best” image obtained for a 20 layer-coated balloon, to avoid overly saturating the image brightness of the samples with thinner coatings. As will be apparent in Results, images of one 20 layer-coated balloon (20LYR1) were slightly over-saturated toward the edges of the balloon. However, this did not impact the resulting quantitative measures, as these measurements were performed mainly along the center axis of the balloon. The fluorescent loadings were quantified based on the histograms of two rectangular regions of interest (ROI) per image (FIG. 1), each of them 100,000 (500×200) pixels in size (total of 12-14 histograms per coated balloon sample) corresponding to 540,832 μm² (0.54 mm²). The ROIs were located along the longitudinal axis as identified using equal distances from the top and bottom edges. The quantification was performed by measuring the mean intensity in each ROI, then averaging the means across all ROIs for a given balloon. Additionally, the overall fluorescence was determined by integrating all brightness values in each histogram and averaging the total brightness across all ROIs for a given balloon. Coating uniformity was determined based on two separate measurements:

1. The first measurement used the standard deviation of each histogram, with higher standard deviations indicating a less uniform distribution. However, as the images were much “brighter” for the balloons containing higher loading, these values may not be used very reliably to compare balloons possessing differing numbers of layers; i.e. the balloons with fewer layers (thus lower intensities overall) will always have smaller standard deviations compared to the balloons with more layers and larger overall brightness.

2. As an alternative for uniformity of distribution, we also quantified the percent of each histogram area that had brightness intensity within ±1-SD, which is likely a better indicator of uniformity of distribution, as it indicates how many pixels (or μm²) have a brightness of Mean±SD.

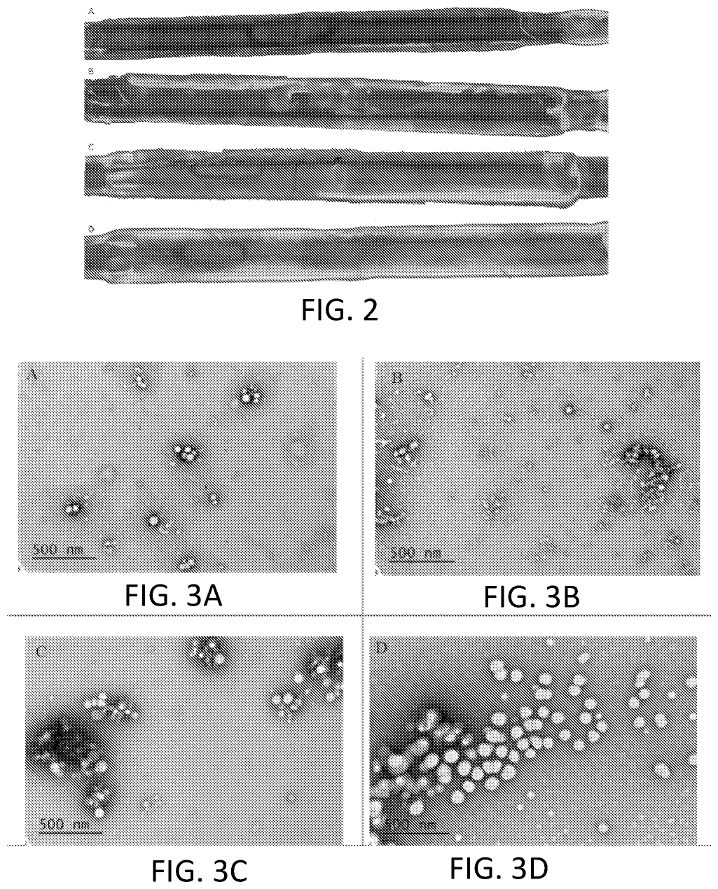

Finally, the fluorescence and brightfield images were stitched together to reconstruct the whole balloon (34), and overlayed for illustration purposes (FIG. 2). All image analyses were performed using Fiji software, and corresponding histogram data was exported into Excel for analysis before plotting using GraphPad Prism version 9 Software (La Jolla, CA).

### Quantification of pNP and Drug Loading Using Gravimetric Analysis and HPLC

Prior to gravimetric analysis, the balloons were clipped from their catheters and were dried under vacuum for 1 hour. Their weights were measured using a Radwag analytical balance. The coating was then eluted using a 1:1 mixture of 90% acetonitrile: dimethylformamide. The coating suspension was acidified with ascorbic acid, vortexed vigorously and centrifuged. The supernatants were stored at −80° C. until HPLC analysis. Finally, the balloons were dried again under vacuum and weighed, so that total coating weights for each balloon could be determined.

### Results and Discussion

### Nanoparticle Characterization

Empty pNP, pNP with entrapped quercetin, pNP with entrapped rhamnazin (3′,7-dimethylquercetin, DMQ), and pNP with quercetin covalently attached to PLGA were spherical in shape with a narrow size distribution (FIG. 3 and Table 1). The particles ranged in size from 64.9±0.8 nm to 161.9±26.6 nm and were monodispersed (polydispersity index (PDI)<0.2). The exception was rhamnazin (3′,7-dimethylquercetin, DMQ) entrapped pNP, which exhibited a PDI of 0.34±0016 (Table 1). Empty pNP and pNP loaded with polyphenols quercetin (3′,7-dimethylquercetin, DMQ) and rhamnazin possessed a small positive charge, with zeta potentials of +6.4-9.3 mV, while pNPs with covalently attached quercetin possessed a negative charge (zeta potential=−29.9±2.4 mV; Table 1).

TABLE 1
Physical Characteristics of the nanodelivery systems

| | Size | | Zeta |
|---|---|---|---|
| | (nm) | PDI | potential (mV) |
| Empty nanoparticles | 64.9 ± 0.8 | 0.129 ± 0.032 | 6.4 ± 0.3 |
| Entrapped | 67.3 ± 1.0 | 0.169 ± 0.003 | 5.9 ± 0.6 |
| quercetin pNPs | | | |
| Entrapped rhamnazin | 161.9 ± 26.6 | 0.342 ± 0.016 | 9.3 ± 0.4 |
| (3′,7- | | | |
| dimethylquercetin, | | | |
| DMQ) pNPs | | | |
| Conjugated quercetin | 106.6 ± 0.8 | 0.050 ± 0.03 | −29.9 ± 2.4 |
| pNPs | | | |

### Drug Release Study

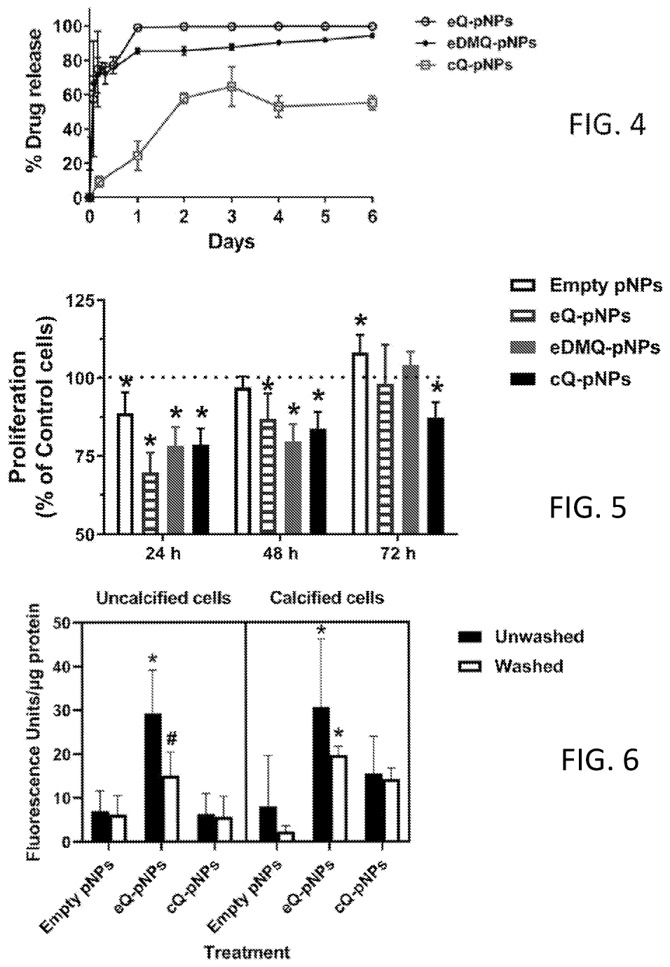

The drug release profile for all 3 entrapped active substances was measured over 6 days. The formulations with entrapped drugs exhibited a burst release within the first day, followed by a more gradual drug release over the remainder of the 6-day period. While entrapped quercetin released rapidly, with 99.7% of the pNP-entrapped quercetin released by day 3, the release was slightly delayed when more hydrophobic alkylated quercetin (rhamnazin (3′,7-dimethylquercetin, DMQ)) was used, with 87.7% released by day 3. The covalent attachment of quercetin to PLGA further delayed its release, as indicated by no burst release, only 64.8% release by day 3, and a gradual release over the remaining 3 days of incubation (FIG. 4).

### Cell Proliferation Assay

RAOSMC were synchronized, stimulated with 10% FBS ±0.4 mg/mL empty or drug-loaded pNP for 2 h and rates of cell proliferation were assessed at 24, 48, and 72 hours as relative rates of BrDU incorporation. These relative rates are expressed as a percent of BrDU incorporation assessed for controls cells receiving no treatment. A two-way ANOVA revealed a significant effect of treatment, time and a significant interaction between treatment and time (FIG. 5), with all pNP treatments significantly reducing RAOSMC proliferation by 11 to 30% at 24 hours. Note that at this initial time point, even empty pNP—pNP(E) —reduced cell proliferation, though the greatest effect was observed for entrapped quercetin (pNP(eQ)). By 48 hours, however, only the drug-containing particles significantly reduced proliferation and by 72 hours, only pNP covalently modified with Q—pNP(cQ) —maintained its inhibitory effect. Of note, by 72 hours, the empty pNPs exhibited a significant increase in RAOSMC proliferation, although it is unclear whether the 8% increase in proliferation observed for this treatment group and time point is of biologic significance.

### Measures of pNP Adhesion

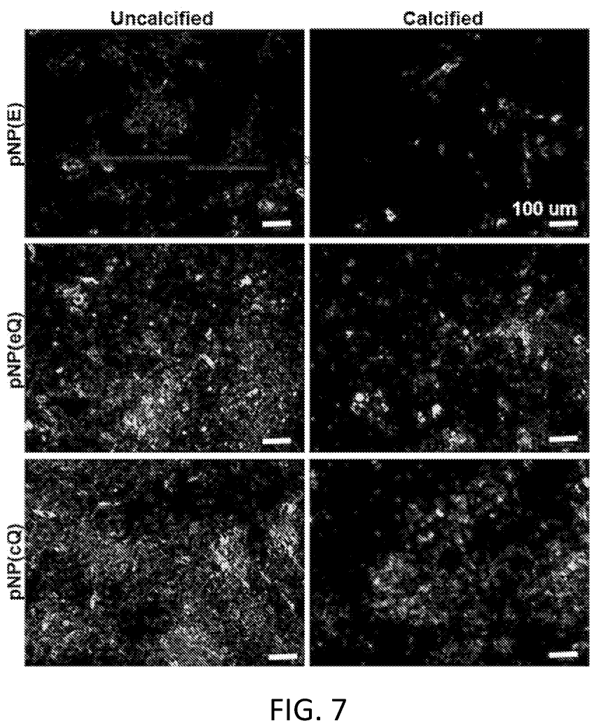

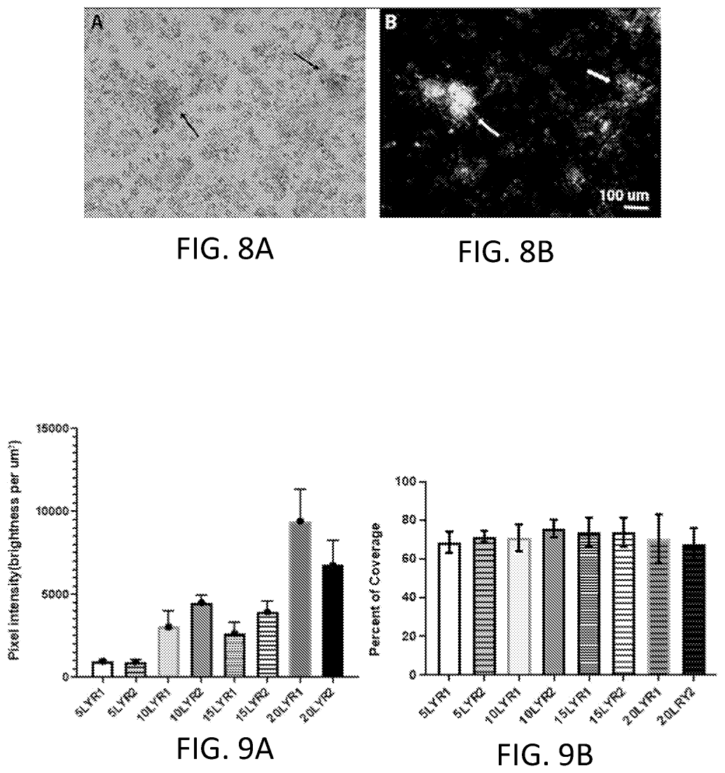

Zeta potential measures showed that the pNP(cQ) possess a negative, rather than a positive charge. Thus, we hypothesized that upon balloon inflation, these particles would exhibit a reduced ability to bind the negatively charged phospholipid bilayer. However, typically, atherosclerotic arteries in PAD are calcified, with tissues accumulating calcium hydroxyapatite. Calcium hydroxyapatite crystals contain both positive and negative ions and its surface charge is highly dependent upon pH (35) Thus, we further hypothesized that given the ionic nature of calcium hydroxyapatite crystals, the pNP may actually exhibit considerable binding to smooth muscle that has become calcified. To test this hypothesis, we allowed the pNP to adhere to RAOSMCs, with one cohort of these cells cultured under calcification conditions. We used fluorescence imaging to quantify pNP adhesion given the ability of quercetin to fluoresce strongly. Results were that pNP containing Q, including eQ and cQ, exhibited greater fluorescence compared to pNP containing no Q (pNP(E); FIGS. 6-7). Fluorescence imaging generally supported this finding, except that we noted for cells treated with pNP containing covalently attached quercetin, strong fluorescence was detected in clusters among cells that were calcified (FIG. 8). We theorize that perhaps these clusters represent pNP(cQ) binding to calcium hydroxyapatite crystals within the smooth muscle cell cultures.

### Loading Based on Fluorescence and the Uniformity of Coating Distribution

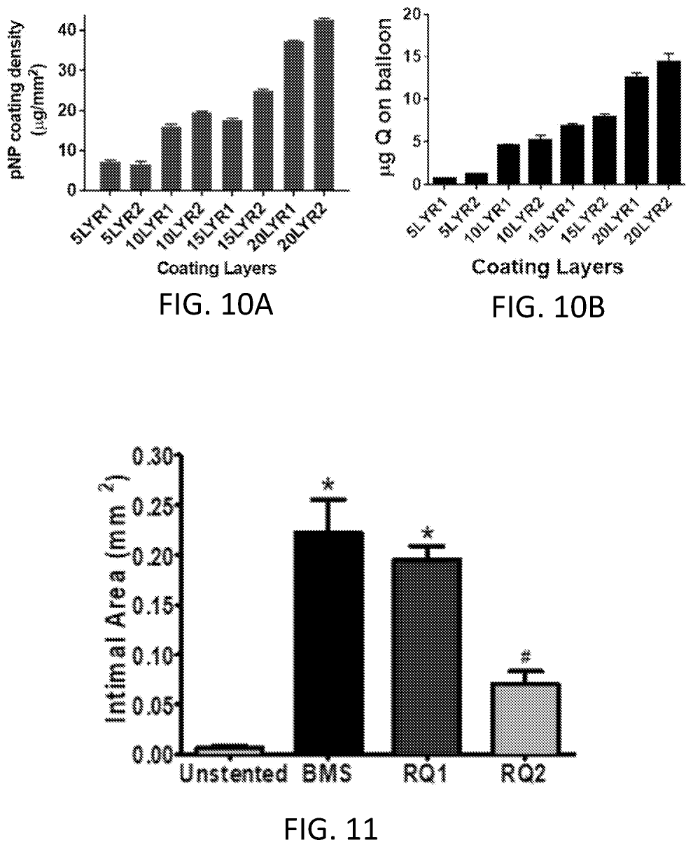

The results of the image analysis indicate that increasing the number of layers increased the fluorescence intensity of the coating, as expected. Note that balloons were named by denoting 1) the number of layers applied (i.e., 5 layers=5LYR), followed by 2) balloon sample number (e.g., 5LYR2=balloon sample 2 coated with 5 layers). There was a clear difference between the samples with 5 layers (5LYR1 and 5LYR2) and the ones with 20 layers (20LYR1 and 20LYR2) (FIG. 9A). However, for balloons with an intermediate number of layers (10LYR1-15LYR1), differences in mean brightness were not clearly distinct from one another, even though both of these had a clearly decreased brightness compared to those with 20 layers, and an increased brightness compared to those with 5-layer balloons. These findings are supported by the drug loading data presented in FIG. 10, where the balloons with 10 and 15 layers show relatively similar amounts of quercetin loadings. These findings may not necessarily indicate an issue with the coating process, as 10LYR2 and 15LYR2 were found to have good coating uniformity across the balloon surface as indicated by both the standard deviations and percent coverage. Overall fluorescence as determined by integrating brightness values over the whole ROIs yielded similar results as the mean values and thus, are not presented here.

Standard deviations for the histograms (FIG. 9A) suggest that the uniformity of coating deposition decreases with an increasing number of layers deposited. However, these findings may be biased by the fact that balloons with fewer coating layers would have much lower overall brightness and thus, smaller standard deviations associated with those mean values. To compensate for differences in standard deviations due to differences in the magnitude of overall brightness, standard deviations were normalized to the mean of each histogram (Table 2). This additional analysis indicates that two of the balloons (10LYR1 and 15LYR1) exhibited a deviation of more than 25% of the mean value, which may be indicative of a lower uniformity of coating compared to the other samples.

TABLE 2
Average fluorescence brightness, absolute standard deviations,
and normalized standard deviations for the coated balloons (max
possible brightness = 12117.4/μm²).

| | Average brightness | Absolute STD | Normalized STD |
|---|---|---|---|
| | (intensity/μm²) | (intensity/μm²) | (% of mean) |
| 5LYR1 | 955.38 | 108.16 | 11.3% |
| 5LYR2 | 908.64 | 137.25 | 15.1% |
| 10LYR1 | 3029.37 | 975.13 | 32.2% |
| 10LYR2 | 4502.90 | 458.34 | 10.2% |
| 15LYR1 | 2547.79 | 661.59 | 26.0% |
| 15LYR2 | 3919.94 | 694.69 | 17.7% |
| 20LYR1 | 9422.61 | 1905.54 | 20.2% |
| 20LYR2 | 6787.45 | 1467.44 | 21.6% |

The second histogram-based measurement of coating uniformity was determined by quantifying the percent area in each histogram that has pixels with brightness (i.e. fluorescence) within ±1-SD of the mean value. This measurement should be independent of absolute pixel brightness in a given ROI. Thus, this value can be used more reliably to compare uniformity between samples with differing numbers of layers (FIG. 9B). Based on these measurements, the percent area covered ranged from 67.6 to 75.8%, which can be considered from good to excellent coverage or uniformity, based on a uniformity scale of: <55% poor, 55-60% moderate, 60-70% good, 70-75% very good, 75-80% excellent, >80% outstanding, with outstanding very rarely occurring in normal image processing of spray-type coatings (a 100% value would indicate all pixels in the ROI having the exact same value, which would be nearly impossible to achieve).

Several of the balloon samples showed cracking of the fluorescent layers, clearly visible in the fluorescent images which may have an unquantified influence on the results of the image analysis, but based on the visual inspection of the images, these were relatively sparse and otherwise small overall. As the coating process occurred in a different location than the fixation on the slide and subsequent imaging, it cannot be ascertained if the cracks are a result of the coating process itself or an artifact introduced by the maybe too rapid drying after coating, or by handling during transport and slide fixation. As the samples with the smallest number of layers (5LYR1 and 5LYR2) did not exhibit any visible cracking, it seems that this phenomenon occurs only for thick layers, which, upon drying, are more prone to cracking.

### Loading of pNP and Quercetin, Assessed Using Gravimetric Analysis Coupled to HPLC

Gravimetric analysis mirrored the results of the fluorescence analyses. pNP coating weights increased nearly linearly with increasing numbers of layers, although coatings with 10 and 15 layers contained more similar amounts of deposited pNP compared to other groups (FIG. 10A). In total, 0.26-1.5 mg of pNP were successfully applied through 5-20 coating layers, respectively (not shown). Adjusting for the surface area of the balloon, this amounted to 7-40 μg/mm² (FIG. 10A).

HPLC analysis of pNP eluted from the balloons revealed a more linear increase in quercetin levels as the coating layers were increased, with total quercetin loading ranging from 0.8-14 μg through 5-20 layers, respectively (FIG. 10B).

### CONCLUSIONS

Peripheral artery disease (PAD) is an inflammatory disease primarily caused by atherosclerosis, which gradually narrows the arterial lumen. Revascularization is considered the first line therapy for symptomatic obstructive PAD (10, 36). Catheter-based percutaneous interventions are an enduring relief for arterial obstruction (36) and are considered the primary method for revascularization (36-38). Restenosis is defined by a reduction in the diameter of the vessel lumen after angioplasty (39). Much research and commercialization effort has been devoted to manufacturing device technologies targeting restenosis (40). The use of polymeric or metallic stents provides better acute results, but these improvements arise at the expense of increased vessel injury (36, 41, 42), with stents commonly resulting in increased risks of thrombosis and stent fracture (43, 44). The need to address the associated risk that comes with stenting led to non-stent-based local drug delivery. Drug-coated balloons are alternative approaches in which the balloon is coated with a thin, active substance surface layer (Byrne et al., 2013). Delayed healing along with vascular toxicity of the anti-proliferative agents applied to the balloon's surface was observed in animal studies after DCB angioplasty (36). In our own prior studies, a nanoparticle delivery system was designed to provide an alternative treatment for PAD, using polyphenols with high therapeutic indices as alternatives to the anti-proliferative agents in commercial products (15). Similar coatings releasing quercetin and resveratrol from drug eluting stents demonstrated outstanding effects in reducing VSMC proliferation, platelet activation and inflammation, while promoting re-endothelization (45, 46). The cationic characteristics of the pNP were provided by addition of a cationic Eudragit RL100 polymer during pNP synthesis. By adjusting the amount of positive charge on the system, the pNP were designed to be biocompatible and biodegradable and proved to meet the specification ideal for cellular uptake and maintaining a continued period of release. The PLGA nanoparticles with pNP(eQ), pNP(eDMQ), as well as quercetin conjugated to PLGA (pNP(cQ), were developed at a size range of 101 nm. All polyphenols were entrapped separately in PLGA pNPs to allow for their comparison. Similar to prior experiments, entrapped quercetin released rapidly in the first 24 hours except that this time the active substance was entrapped separately in pNPs not together with RESV in its methoxylated form (15). However, covalent attachment of quercetin delayed its release as indicated by no burst release and a more protracted profile. The methoxylated derivative of quercetin (rhamnazin (3′,7-dimethylquercetin, DMQ)) with increased hydrophobicity provided a slightly more sustained release of quercetin, although was not as protracted as pNPs possessing covalently-attached Q. In the latter case, release was sustained for a total of 6 days, which is beneficial since vascular healing, as well as the cellular events contributing to restenosis, begin within the first 7 days (47).

In this experiment an ultrasonic coating method was used that allowed our pNP entrapment system to generate a uniform coating. This coating technique will hopefully minimize non-specific release of drug into the blood and enhance the long-term retention of drug within vascular tissue, but such specifications will be addressed in future animal experiments.

In summary, a key parameter for a successful DCB is delivery of therapeutic levels of drug at biologically appropriate time points within a critical time window after endovascular intervention. The synthesized PLGA-based pNP system proved to be biocompatible with a size range required for endocytosis and provided an extended period of release. Importantly, brief application with pNPs containing covalently-attached Q demonstrated an ability to reduce VSMC proliferation at least through 72 hours. Studies utilizing a balloon angioplasty model in small animals aimed at testing the pharmacokinetics of drug delivery to the vascular wall will be required for further development.

### REFERENCES

1. Song P, Rudan D, Zhu Y, Fowkes F J I, Rahimi K, Fowkes F G R, et al. Global, regional, and national prevalence and risk factors for peripheral artery disease in 2015: an updated systematic review and analysis. The Lancet Global Health. 2019; 7(8):e1020-e30.
2. Patel M R, Conte M S, Cutlip D E, Dib N, Geraghty P, Gray W, et al. Evaluation and treatment of patients with lower extremity peripheral artery disease: Consensus definitions from peripheral academic research consortium (PARC). Journal of the American College of Cardiology. 2015; 65(9):931-41.
3. Jaff M R, Cahill K E, Yu A P, Birnbaum H G, Engelhart Luella M M. Clinical Outcomes and Medical Care Costs Among Medicare Beneficiaries Receiving Therapy for Peripheral Arterial Disease. Annals of Vascular Surgery. 2010; 24(5):577-87.
4. Beckman J A, Findeiss L K, Golzarian J, Gornik H L, Halperin J L, Jaff M R, et al. This article is copublished in Circulation, Catheterization and Cardiovascular Interventions, the Journal of Vascular Surgery, and Vascular Medicine. Jac. 2011; 58(19):2020-45.
5. Fowkes F G R, Aboyans V, Fowkes F J I, McDermott M M, Sampson U K A, Criqui M H.

Peripheral artery disease: epidemiology and global perspectives. Nature Reviews Cardiology. 2017; 14(3):156-70.
6. Marrocco C J, Bush H R L. Peripheral arterial disease. High Risk Diabetic Foot: Treatment and Prevention. 2010; 358:1-8.
7. Konishi A, Mitsutake Y, Ho M, Handa N, Koike K, Mochizuki S, et al. Patient and lesion characteristics in late/very late stent thrombosis with everolimus-eluting stents from real-world adverse event reporting. Journal of Cardiology. 2019:3-8.
8. Tendera M, Aboyans V, Bartelink M L, Baumgartner I, Clment D, Collet J P, et al. ESC Guidelines on the diagnosis and treatment of peripheral artery diseases. European Heart Journal. 2011; 32(22):2851-906.
9. Barrett C, Barshes N R, Corriere M A, Drachman D E, Fleisher L A, Gerry Fowkes F R, et al. 2016 AHA/ACC Guideline on the Management of Patients With Lower Extremity Peripheral Artery Disease. Circulation. 2017; 135:726-79.
10. Norgren L, Hiatt W R, Dormandy J A, Nehler M R, Harris K A, Fowkes F G, et al. Inter-society consensus for the management of peripheral arterial disease. International angiology: a journal of the International Union of Angiology. 2007; 26(2):81-157.
11. Urasawa K, Sato K, Koshida R, Honma Y. [Endovascular therapy for peripheral arterial disease]. Nippon rinsho Japanese journal of clinical medicine. 2011; 69(2):318-21.
12. Analysis A E-b. Stenting for peripheral artery disease of the lower extremities: An evidence-based analysis2010. 1-88 p.
13. Thukkani A K, Kinlay S. Endovascular Intervention for Peripheral Artery Disease. Circulation Research. 2015; 116(9):1599-613.
14. Rogers J H, Laird J R. Overview of new technologies for lower extremity revascularization. Circulation. 2007; 116(18):2072-85.
15. Dugas T R, Brewer G, Longwell M, Fradella T, Braun J, Astete C E, et al. Nanoentrapped polyphenol coating for sustained drug release from a balloon catheter. Journal of Biomedical Materials Research—Part B Applied Biomaterials. 2019; 107(3):646-51.
16. Ali R M, Abdul Kader MASK, Wan Ahmad W A, Ong T K, Liew H B, Omar A F, et al. Treatment of Coronary Drug-Eluting Stent Restenosis by a Sirolimus- or Paclitaxel-Coated Balloon. JACC: Cardiovascular Interventions. 2019; 12(6):558-66.
17. Van Den Berg J C. In-stent restenosis management: The best is yet to come. Journal of Cardiovascular Surgery. 2017; 58(4):508-17.
18. Goyal S N, Bharti S, Krishnamurthy B, Agrawal Y, Ojha S K, Arya D S. Impact of metabolic syndrome on re-stenosis development: Role of drug-eluting stents. Diabetes and Vascular Disease Research. 2012; 9(3):177-88.
19. Werk M, Langner S, Reinkensmeier B, Boettcher H F, Tepe G, Dietz U, et al. Inhibition of restenosis in femoropopliteal arteries. Paclitaxel-coated versus uncoated balloon: Femoral paclitaxel randomized pilot trial. Circulation. 2008; 118(13):1358-65.
20. Tepe G, Laird J, Schneider P, Brodmann M, Krishnan P, Micari A, et al. Drug-coated balloon versus standard percutaneous transluminal angioplasty for the treatment of superficial femoral and popliteal peripheral artery disease 12-month results from the IN.PACT SFA randomized Trial. Circulation. 2015; 131(5):495-502.
21. Tepe G, Zeller T, Albrecht T, Heller S, Schwarzwslder U, Beregi J P, et al. Local delivery of paclitaxel to inhibit restenosis during angioplasty of the leg. New England Journal of Medicine. 2008; 358(7):689-99.
22. Cortese B, Granada J F, Scheller B, Schneider P A, Tepe G, Scheinert D, et al. Drug-coated balloon treatment for lower extremity vascular disease intervention: An international positioning document. European Heart Journal. 2016; 37(14):1096-103.
23. Doshi M, Sojitra P, Shah D, Dani S, Abizaid A. Technical Insights on Drug-Coated Balloons II. Drug-Coated Balloons. 2019:45-57.
24. Marzullo R, Aprile A, Clementi F, Stella P, Modena M G, Sangiorgi G M. Paclitaxel eluting balloon: From bench to bedside. Minerva Cardioangiologica. 2009; 57(5):597-609.
25. McFadden E P, Stabile E, Regar E, Cheneau E, Ong A T L, Kinnaird T, et al. Late thrombosis in drug-eluting coronary stents after discontinuation of antiplatelet therapy. Lancet. 2004; 364(9444):1519-21.
26. Farb A, Heller P F, Shroff S, Cheng L, Kolodgie F D, Carter A J, et al. Pathological analysis of local delivery of paclitaxel via a polymer-coated stent. Circulation. 2001; 104(4):473-9.
27. Fda. Aug. 7, 2019 UPDATE: Treatment of Peripheral Arterial Disease with Paclitaxel-Coated Balloons and Paclitaxel-Eluting Stents Potentially Associated with Increased Mortality. 2019; 57:13-5.
28. Yurdagul A, Kleinedler J J, McInnis M C, Khandelwal A R, Spence A L, Orr A W, et al. Resveratrol promotes endothelial cell wound healing under laminar shear stress through an estrogen receptor-α-dependent pathway. American Journal of Physiology—Heart and Circulatory Physiology. 2014; 306(6):797-806.
29. Narula N, Olin J W, Narula N. Pathologic disparities between peripheral artery disease and coronary artery disease. Arteriosclerosis, thrombosis, and vascular biology. 2020; 40(9):1982-9.
30. Karwowski W, Naumnik B, Szczepanski M, Mysliwiec M. The mechanism of vascular calcification—a systematic review. Medical science monitor: international medical journal of experimental and clinical research. 2012; 18(1):RA1.
31. Fitzgerald P J, Ports T A, Yock P. Contribution of localized calcium deposits to dissection after angioplasty. An observational study using intravascular ultrasound. Circulation. 1992; 86(1):64-70.
32. Fanelli F, Cannavale A, Gazzetti M, Lucatelli P, Wlderk A, Cirelli C, et al. Calcium burden assessment and impact on drug-eluting balloons in peripheral arterial disease. Cardiovascular and interventional radiology. 2014; 37(4):898-907.
33. Trion A, van der Laarse A. Vascular smooth muscle cells and calcification in atherosclerosis. American heart journal. 2004; 147(5):808-14.
34. Preibisch S, Saalfeld S, Tomancak P. Globally optimal stitching of tiled 3D microscopic image acquisitions. Bioinformatics. 2009; 25(11):1463-5.
35. Harding I S, Rashid N, Hing K A. Surface charge and the effect of excess calcium ions on the hydroxyapatite surface. Biomaterials. 2005; 26(34):6818-26.
36. Byrne R A, Joner M, Alfonso F, Kastrati A. Drug-coated balloon therapy in coronary and peripheral artery disease. 2013.
37. Wiseman J T, Fernandes-Taylor S, Saha S, Havlena J, Rathouz P J, Smith M A, et al.

Endovascular Versus Open Revascularization for Peripheral Arterial Disease. Ann Surg. 2017; 265(2):424-30.
38. AbuRahma A F. When Are Endovascular and Open Bypass Treatments Preferred for Femoropopliteal Occlusive Disease? Annals of Vascular Diseases. 2018; advpub.
39. Omeh D J, Shlofmitz E. Restenosis. StatPearls [Internet]. 2020.
40. Curcio A, Torella D, Cuda G, Coppola C, Faniello M C, Achille F, et al. Effect of stent coating alone on in vitro vascular smooth muscle cell proliferation and apoptosis. American Journal of Physiology-Heart and Circulatory Physiology. 2004; 286(3):H902-H8.
41. Kuntz R E, Baim D S. Defining coronary restenosis. Newer clinical and angiographic paradigms. Circulation. 1993; 88(3):1310-23.
42. Costa M A, Simon D I. Molecular basis of restenosis and drug-eluting stents. Circulation. 2005; 111(17):2257-73.
43. Rocha-Singh K J, Bosiers M, Schultz G, Jaff M R, Mehta M, Matsumura J S, et al. A single stent strategy in patients with lifestyle limiting claudication: 3-year results from the Durability II trial. Catheterization and Cardiovascular Interventions. 2015; 86(1):164-70.
44. Scheinert D, Scheinert S, Sax J, Piorkowski C, Braunlich S, Ulrich M, et al. Prevalence and clinical impact of stent fractures after femoropopliteal stenting. Journal of the American College of Cardiology. 2005; 45(2):312-5.
45. Kleinedler J J, Foley J D, Orchard E A, Dugas T R. Novel nanocomposite stent coating releasing resveratrol and quercetin reduces neointimal hyperplasia and promotes re-endothelialization. Journal of Controlled Release. 2012; 159(1):27-33.
46. Kleinedler J J, Foley J D, Alexander J S, Roerig S C, Hebert V Y, Dugas T R. Synergistic effect of resveratrol and quercetin released from drug-eluting polymer coatings for endovascular devices. Journal of Biomedical Materials Research Part B: Applied Biomaterials. 2011; 99(2):266-75.
47. Kenagy R D. 7⋅Biology of Restenosis and Targets for Intervention. Mechanisms of Vascular. 2011:115.

### Example 2

### Potential Efficacy of RQ-Eluting Medical Device:

Initial studies demonstrated that resveratrol and quercetin synergized to significantly reduce VSMC proliferation in in vitro models.^{1,2} RQ-eluting coating could have utility on endovascular devices so we tested the efficacy of an RQ coating on a drug eluting stent (DES) in a rodent model of angioplasty and stenting. The RQ-DES showed dramatic efficacy. In brief, mini-stents were coated with a biostable polymer containing 50:25 μg/cm² R:Q (designated as RQ1) and 150:75 μg/cm² (RQ2). The stents were implanted into male and female rats (n=6/gp) and at 28 d, the stented arteries were analyzed. The results were dramatic.

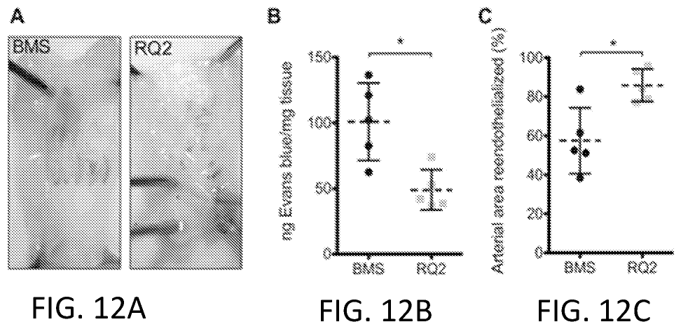

Implantation of bare metal stents (BMS) resulted in a profound increase in intimal area compared to contralateral unstented arteries (FIG. 11). Also, % luminal stenosis, defined as area of lumen occupied by neointima, increased from 1.4% in unstented arteries to 30% in stented arteries. Note that our measures of % stenosis mirror that reported by Virmani et al, who utilized the rabbit iliac artery model.³ What was exciting is that the RQ2-DES reduced intimal areas by 70% compared to BMS, and intimal areas in the RQ2-DES group were not different from that of contralateral, unstented arteries. In addition, arteries stented with BMS demonstrated moderate inflammation, including luminal neutrophil adhesion.⁴ Luminally-attached neutrophils tended to be less for RQ2 than for RQ1 (2.3±1.5 vs. 5.8±3.0; p<0.05) or BMS (2.3±1.5 vs. 5.0±1.3), respectively. Moreover, the RQ2 group exhibited less immunostaining for macrophages compared to BMS (7.2±5.9 vs. 17.9±4.5 macrophage/section; p<0.05). A separate set of rats were injected with Evans blue dye and sacrificed at 10 d. Evans blue dye is taken up into areas lacking an endothelium. Endothelial re-growth was only ˜58% complete in the BMS group. Importantly, the extent of re-endothelialization was 50% greater in arteries implanted with RQ2 compared to BMS (FIG. 12). To confirm these results, the dye was extracted from the tissue and quantified. Consistent with greater endothelial coverage, 50% less Evans blue dye accumulated into the carotids of animals receiving the RQ2 compared with BMS (FIG. 12).

### REFERENCES FOR EXAMPLE 2

1. Kleinedler, J J, Foley, J D, Alexander, J S, Roerig, S C, Hebert, V Y, and Dugas, T R. Synergistic effect of resveratrol and quercetin released from drug-eluting polymer coatings for endovascular devices. J. Biomed. Mater. Res. Pt B. 2011. 99(2):266-75.
2. Khandelwal A R, Hebert V Y, Kleinedler J J, Rogers L K, Ullevig S L, Asmis R, Shi R, Dugas T R. Resveratrol and quercetin interact to inhibit neointimal hyperplasia in mice with a carotid injury. J Nutr. 2012. 142(8):1487-94.
3. Ribichini F, Joner M, Ferror V, Finn A V, Crimins J, Nakazawa G, Acampado E, Kolodgie F D, Vassanelli C, and Virmani R. Effects of oral prednisone after stenting in a rabbit model of established atherosclerosis. J Amer Coll Cardiol. 2007. 50:176-185.
4. Kleinedler, J J, Foley, J D, Orchard, E A, and Dugas, T R. Novel nanocomposite stent coating releasing resveratrol and quercetin reduces neointimal hyperplasia and promotes re-endothelialization. J. Control. Rel. 2012. 159:27-33.

What is claimed is:

1. A composition comprising a polymeric nanoparticle made of a polymer having an agent is covalently bonded to the polymer.

2. The composition of claim 1, wherein the polymer is selected from the group consisting of poly(lactide), poly(glycolide), poly(lactide-co-glycolide), poly(caprolactone), poly(lactide-co-caprolactone), poly(glycolide-co-caprolactone), and poly(D,L-lactide-co-glycolide-co-ε-caprolactone).

3. The composition of claim 1, wherein the agent is selected from the group consisting of resveratrol, pharmaceutically acceptable salts, and pharmaceutically acceptable derivatives thereof, or quercetin, pharmaceutically acceptable salts, and pharmaceutically acceptable derivatives thereof, rhamnazin, pharmaceutically acceptable salts, and pharmaceutically acceptable derivatives thereof, or a combination of any of these.

4. The composition of claim 4, wherein the polymer is a poly(lactide-co-glycolide).

5. The composition of claim 1, wherein agent is quercetin, pharmaceutically acceptable salts, and pharmaceutically acceptable derivatives thereof.

6. The composition of claim 1, comprising nanoparticles having a diameter of about 25 nm to about 300 nm.

7. The composition of claim 1, further comprising a second agent encapsulated by the polymer of the polymeric nanoparticle.

8. The composition of claim 1, wherein second agent is resveratrol, pharmaceutically acceptable salts, and pharmaceutically acceptable derivatives thereof.

9. The composition of claim 1, wherein the polymeric nanoparticle is charged.

10. The composition of claim 9, wherein the polymeric nanoparticle is cationic.

11. The composition of claim 9, wherein the polymeric nanoparticle is anionic.

12. The composition of claim 9, wherein the polymeric nanoparticles have a pH-independent zeta potential of about 0.35 mV to about 0.60 mV.

13-18. (canceled)

19. A composition comprising a first polymeric nanoparticle made of a first polymer having a first agent covalently bonded to the polymer and a second polymeric nanoparticle made of a second polymer, where the second polymer encapsulates a second agent.

20. The composition of claim 19, wherein the first polymer and the second polymer are independently selected from the group consisting of poly(lactide), poly(glycolide), poly(lactide-co-glycolide), poly(caprolactone), poly(lactide-co-caprolactone), poly(glycolide-co-caprolactone), and poly(D,L-lactide-co-glycolide-co-ε-caprolactone).

21. The composition of claim 19, wherein the first agent and the second agent are independently selected from the group consisting of resveratrol, pharmaceutically acceptable salts, and pharmaceutically acceptable derivatives thereof, or quercetin, pharmaceutically acceptable salts, and pharmaceutically acceptable derivatives thereof, rhamnazin, pharmaceutically acceptable salts, and pharmaceutically acceptable derivatives thereof, or a combination of any of these.

22. The composition of claim 19, wherein the first polymer, second polymer, or both are a poly(lactide-co-glycolide).

23. The composition of claim 19, wherein second agent is resveratrol, pharmaceutically acceptable salts, and pharmaceutically acceptable derivatives thereof.

24. The composition of claim 19, wherein first agent is quercetin, pharmaceutically acceptable salts, and pharmaceutically acceptable derivatives thereof.

25. The composition of claim 19, comprising nanoparticles having a diameter of about 25 nm to about 300 nm.

26. The composition of claim 19, wherein the polymeric nanoparticle is charged.

27-36. (canceled)
